(19) United States
(12) Patent Application Publication
(10) Pub. No.: US 2025/0170188 A1
(43) Pub. Date: May 29, 2025

(54) BACILLUS CLAUSII STRAIN, COMPOSITIONS THEREOF, AND METHODS OF USE

(71) Applicant: DEERLAND PROBIOTICS & ENZYMES, INC., Kennesaw, GA (US)

(72) Inventor: John DEATON, Kennesaw, GA (US)

(73) Assignee: DEERLAND PROBIOTICS & ENZYMES, INC., Kennesaw, GA (US)

(21) Appl. No.: 18/712,476

(22) PCT Filed: Dec. 8, 2022
(86) PCT No.: PCT/US2022/081161

§ 371 Date: May 22, 2024

(30) Foreign Application Priority Data
Dec. 8, 2021 (IE) 2021/0209

(51) Int. Cl. A61K 35/742

(52) U.S. Cl. CPC A61K 35/742

(57) ABSTRACT

The invention provides a Bacillus clausii strain comprising a purified microbial population that comprises one or more bacteria with a gyrB that shares at least 97% identity with SEQ ID NO: 1; and/or that comprises one or more bacteria with a 16S rRNA that shares at least 97% identity with SEQ ID NO: 2. Optionally, the Bacillus clausii strain shares at least 97% identity with SEQ ID NO: 3. Compositions and methods of using the Bacillus clausii are also part of this invention.

### FIELD OF THE INVENTION

This invention relates to a new Bacillus clausii strain, which alone or in combination with other Bacilli strains can be used as probiotics or together with a prebiotic and a symbiotic. The invention also relates to a composition such as a pharmaceutical composition, dairy product, functional food, nutraceutical, dietary supplement, and product for personal care comprising the new Bacillus clausii strain alone or in combination with other strain(s), as well as use of the strain for prevention or treatment of gastrointestinal, urinary tract, vaginal, and other infections and diseases, and other uses.

### BACKGROUND OF THE INVENTION

Probiotics are live microorganisms or microbial mixtures administered to improve the patient's microbial balance, particularly the environment of the respiratory and gastrointestinal tract. Bacillus strains have been employed for the treatment of respiratory infections, prevention of diarrhoea, as well as, for the treatment of immuno-related diseases (Elshaghabee et al., 2017).

The normal intestinal flora is dominated by various bacterial species, which produce substances that help control the growth of pathogens. Dysbiosis is a condition that is characterized by a decrease of the certain bacterial species and an increased growth of pathogenic bacteria. Dysbiosis has been associated with the development of periodontal disease, inflammatory bowel disease, and chronic fatigue syndrome. Some studies have suggested patients with dysbiosis may have an increased risk of developing metabolic and cardiac disorders (Chan et al., 2013).

By administering probiotic Bacilli, it is possible to regenerate the intestinal flora of men and women with recurrent episodes of dysbiosis. Dysbiosis is a common gastrointestinal problem. Dysbiosis caused by Escherichia coli is also a common problem (Chan et al., 2013).

The presence of Bacilli is important for the maintenance of the intestinal microbial ecosystem. Bacilli have been shown to possess inhibitory activity toward the growth of pathogenic bacteria such as Listeria monocytogenes, Escherichia coli, Salmonella spp. and others (Yilmaz et al., 2005). This inhibition could be due to the production of inhibitory compounds such as organic acids, hydrogen peroxide, bacteriocins or reuterin or to competitive adhesion to the epithelium (Abriouel et al., 2010).

Bacilli have also been examined as a treatment of respiratory tract infections (Marseglia et al., 2007). For example, the installation of Bacilli, and stimulation of indigenous organisms has been employed to prevent recurrence of urinary tract infections (Marseglia et al., 2007). The role of Bacilli in preventing intestinal infections has also been investigated.

### DESCRIPTION OF RELATED ART

The importance of Bacilli as probiotics has been described in the literature.

Hyronimus et al., 2000 discloses the screening of probiotic activities of a number of Bacilli strains by in vitro techniques and evaluation of the colonization ability of thirteen selected strains in humans. The strains were examined for resistance to pH 2.5 and 0.3% Oxgall adhesion to Caco-2 cells and antimicrobial activities against enteric pathogenic bacteria (Khochamit et al., 2015). Bacilli have been shown to possess the primary requirement of GIT stress tolerance, besides having good adhesion and bio-therapeutic properties (Thakur et al., 2016).

Pharmaceutical compositions of Bacilli known in the art are not sufficiently efficient in recolonizing in vivo i.e., mammalian microbial ecosystems, and there is, therefore, a need to find Bacilli with an inherent ability to recolonize upon administering the Bacilli in the form of a pharmaceutical composition, a nutraceutical, a dairy product, a functional food or absorbent product. Bacilli isolated from soil may have the ability to recolonize in vivo upon administration because of their inherent ability to survive in the human microbial ecosystem. It is often a cumbersome process to identify Bacilli strains with enhanced abilities to colonize upon administration and it is therefore important to select useful test systems to predict their in vivo ability to colonize.

In the literature, there seems to be a large variation in the reported in vitro adherence of probiotic strains. This variation indeed reflects biological differences between strains, but certainly also depends on experimental conditions. Moreover, there also seems to be variation with regard to how to measure the adherence. In some cases, it may be argued that an in vitro experiment only serves as a means to estimate the in vivo ability to colonize by adherence to epithelial cells.

Despite being long considered soil microorganisms, Bacillus spp. have been used for more than 50 years in the form of fermentation products or spore-based supplements (Cutting et al., 2011). Bacilli, being ubiquitous in nature, consistently enter the gastrointestinal and respiratory tracts of healthy people through food, water, and air (Benno & Mitsuoka, 1986). They have been isolated from the gut and can reach up to 10⁷ CFU/g and hence are considered to be one of the dominant components of the normal gut microbiota (Lakshmi et al., 2017). More recently, strains of Bacillus clausii have been isolated in order to provide more specific functions and its safety has been evaluated. Bacillus clausii has been previously used in diarrheal patients (Sudha et al., 2013, Horosheva et al., 2014) and children with recurrent respiratory infections (Marseglia et al., 2007) with no adverse events reported. Though the countries and strains are not specified, Bacillus clausii has been commercialized in 55 countries around the world (Nista et al. 2004; Gabrielli et al. 2009). The literature review for Bacillus clausii showed no adverse events related to the probiotic and the worldwide presence of bacteria in different countries supplements the narrative of its safety for human consumption.

In summary, Bacilli strains with probiotic capabilities should be able to adhere to other suitable cells, such as the cell line Caco-2 cells. Moreover, it is also desirable that the Bacilli strains with probiotic capabilities show in vitro inhibitory activity against other bacterial species, produce acid after growth in liquid culture and/or produce hydrogen peroxide.

### SUMMARY OF THE INVENTION

It is an object of the present invention to provide strains and compositions as described throughout this application such as pharmaceutical formulations or absorbent products of suitable probiotic strains such as Bacilli strains with desirable properties. In an embodiment, the present invention concerns the Bacillus clausii strain CSI08 alone or in combination with other Bacilli strains such as Bacillus megaterium strain MIT411 (disclosed and claimed in corresponding PCT Application PCT/US2022/xxxxx claiming priority from Irish Patent Application No. 2021/0211, whose contents are incorporated herein in their entirety) and Bacillus coagulans strain CGI314 (disclosed and claimed in corresponding PCT Application PCT/US2022/xxxxx claiming priority from Irish Patent Application No. 2021/0210, whose contents are incorporated herein in their entirety). In an embodiment, these strains have similar or essentially the same advantageous properties e.g. the ability to colonize by adherence to mucosal membranes/surfaces and which are therefore suited for the treatment or prevention of infections or diseases of the vaginal, urinary-tract, gastrointestinal, naso-sinal, pharyngeal, esophageal, oral, and/or other areas of the body with mucosal membranes, as well as, the treatment or prevention of infections or diseases of the skin and/or other areas of the body having epithelium; immune health, protection against oxidative stress, cleansing and detoxification, metabolic health and cardiovascular health amongst others such as providing antimicrobial activity, anti-inflammatory activity, suppression of pro-inflammatory response, activating and/or provoking immune response eg. by stimulating macrophages, providing immunoprotection, aiding in digestion and/or fermentation for instance in the gut, producing branched amino acids, essential amino acids and group B vitamins, maintaining healthy gut and/or skin, protection of mucosal and other epithelial tissues from toxic agents, decreasing incidence of loose stools, improving the gut-brain axis, and treating and/or preventing dysbiosis and its effects such as periodontal disease, inflammatory bowel disease, chronic fatigue syndrome, metabolic disorders, cardiac disorders, respiratory tract infections, urinary tract infections, GI infections, and diarrhea; and restoring normal and/or healthy flora. In an embodiment, the present invention allows the use of Bacillus clausii strain CSI08 and compositions for use in fecal transplants.

Gastrointestinal diseases include, but are not limited to, treating gastrointestinal irregularity in an individual, wherein the individual has at least one 24-hour episode per month of bowel movements measuring 1 or 2 on the Bristol Stool Scale (i.e. treating constipation; or wherein the individual has at least one 24-hour episode per month of bowel movements measuring 6 to 7 on the Bristol Stool Scale (tending towards diarrhea), wherein the frequency of the individual's 24-hour episodes per month of bowel movements measuring 1 or 2 (or 6 to 7) on the Bristol Stool Scale decreases.

Also included is a method of restoring gastrointestinal regularity in an individual, wherein the individual has at least one 24-hour episode per month of bowel movements measuring 1 or 2; or 6 to 7 on the Bristol Stool Scale, wherein the frequency of 24-hour periods of the individual's bowel movements measuring from 3 to 5 on the Bristol Stool Scale increases.

The invention further includes maintaining healthy gut microflora, with Bacillus-containing composition(s). The Bacillus-containing composition(s) can be used as probiotic supplementation of the gastrointestinal microflora, and may compete with or otherwise discourage pathogenic bacteria in the gut such as Escherichia coli, Listeria monocytogenes, Salmonella spp.

Another object of the present invention is to provide pharmaceutical formulations with an increased ability to colonize by adherence to the mucosal membrane by employing mucous adhesive excipients.

It is a further object of the present invention to provide vaginal formulations with an increased ability to suppress the growth of Candida albicans and Gram-negative pathogenic bacteria.

It is yet another objective of the present invention to provide compositions such as dairy products, nutraceutical products and functional foods comprising Bacillus clausii strain CSI08 alone or in combination with other Bacilli strains such as Bacillus megaterium strain MIT411 and/or Bacillus coagulans strain CGI314, in an embodiment, having essentially the same properties, in an embodiment having the ability to colonize mucosal membranes and therefore adapt to treatment or prevention of vaginal infections, urinary-tract infections and gastrointestinal diseases. Compositions of the present invention may be administered for 1 dose, 1 day, 1 day to 1 week, 1 day to 1 month, 1 month to 45 days, 45 days to 2 months, 3 months, 6 months, 1 year, or more, including any timeframe identified and/or falling within these ranges.

### FIGURES

### In the Drawings:

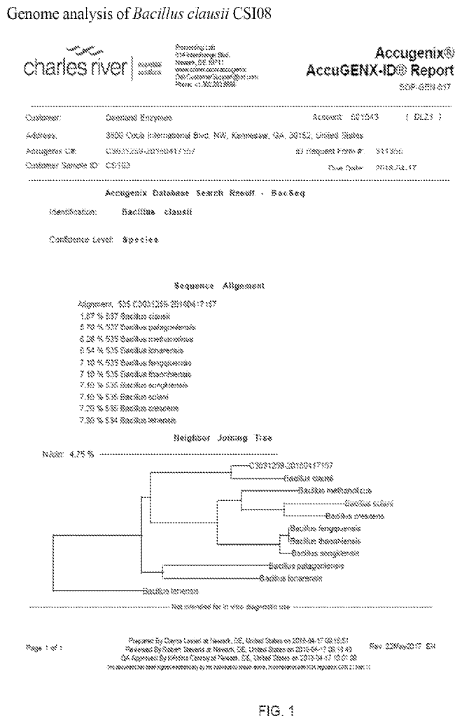

FIG. 1 illustrates the genome analysis of Bacillus clausii CSI08.

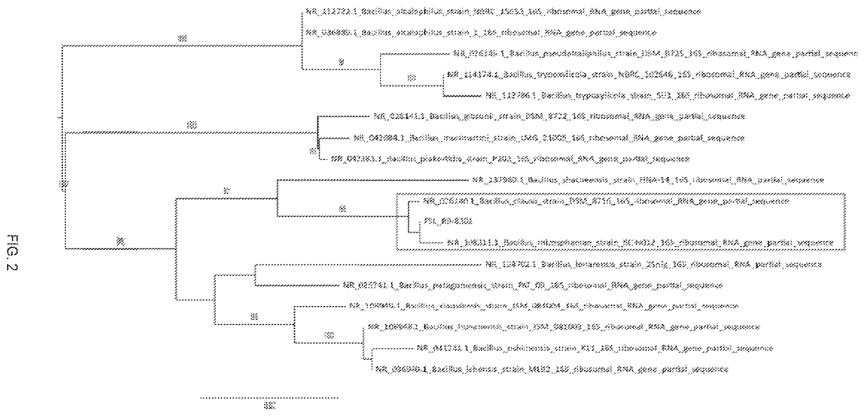

FIG. 2 illustrates the phylogenetic tree (16S) of Bacillus spp, arranged in clades.

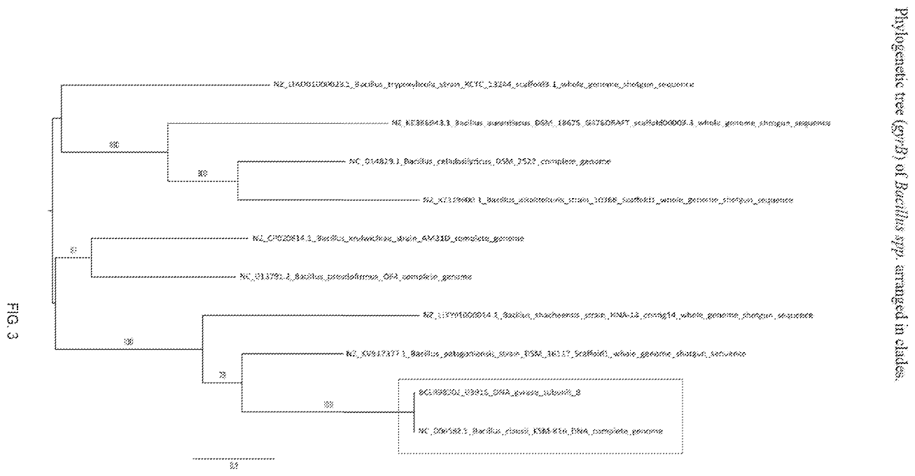

FIG. 3 illustrates the phylogenetic tree (gyrB) of Bacillus spp., arranged in clades.

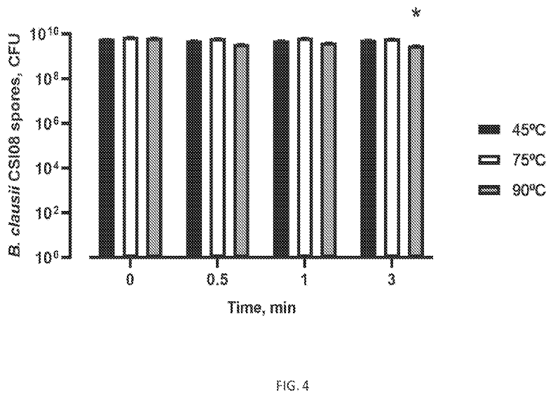

FIG. 4 shows the stability of MuniSpore in PBS during a pasteurization process.

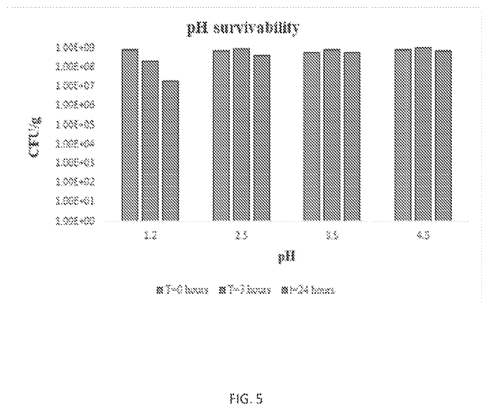

FIG. 5 shows pH Survivability of B. clausii CSI08 spores at 0-, 3-, and 24-hour timepoints.

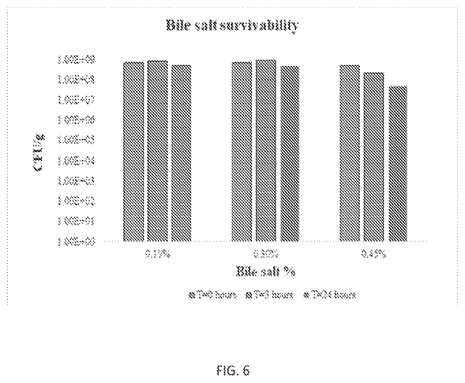

FIG. 6 shows bile salt survivability of B. clausii CSI08 spores at 0-, 3-, and 24-hour timepoints.

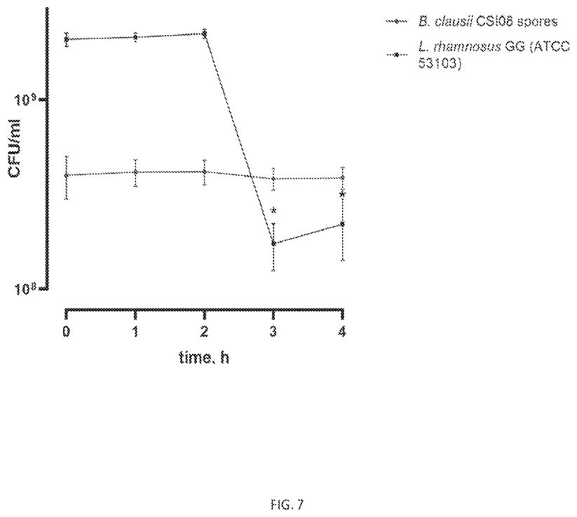

FIG. 7 shows survival of Bacillus clausii CSI08 spores and L. rhamnosus GG under gastric and small intestinal digestion conditions simulated in vitro.

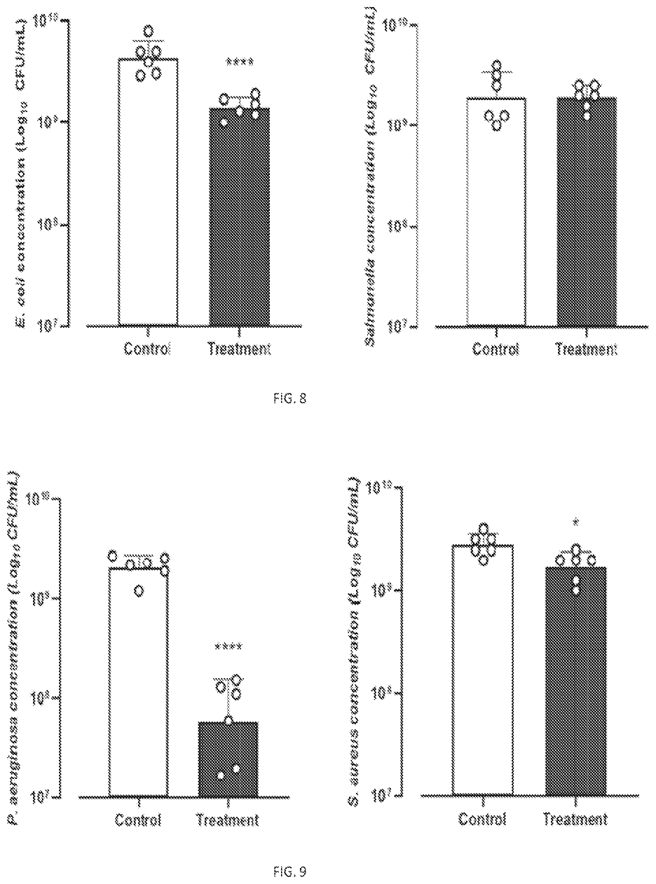

FIGS. 8 and 9 show antimicrobial activity of Munispore (B. clausii CSI08) in liquid culture.

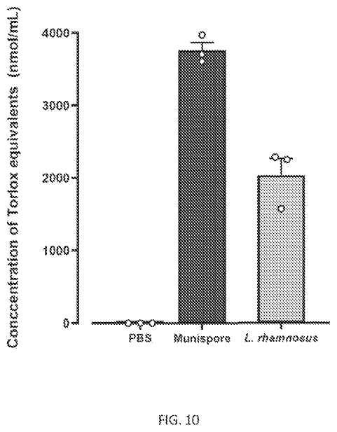

FIG. 10 shows total antioxidant capacity of B. clausii CS108 and L. rhamnosus GG.

FIG. 11 shows the cytoprotective effect of the vegetative form B. clausii CSI08 on H₂O₂-exposed epithelium.

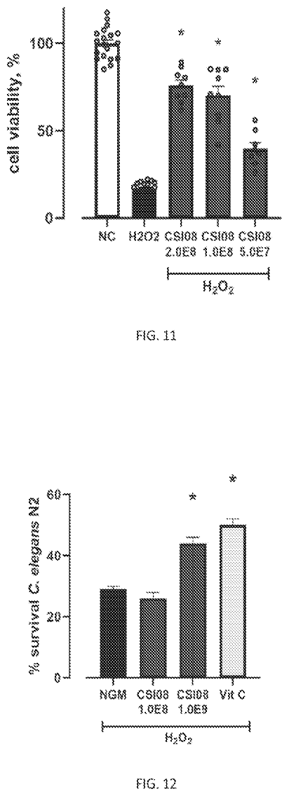

FIG. 12 shows the survival rate of C. elegans N2 fed with 10⁸ and 10⁹ CFU/ml B. clausii CSI08, followed by oxidative stress caused by H₂O₂. NGM-control-fed nematodes; Vit C-positive control.

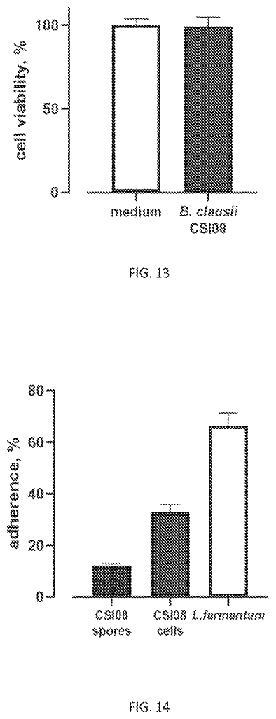

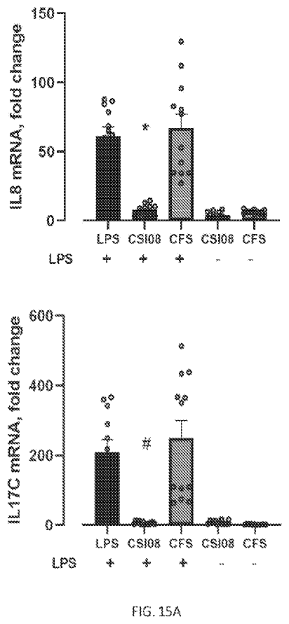

FIG. 13 shows the effect of vegetative cells B. clausii CSI08 on HT-29 cell viability. B. clausii CSI08 compared with untreated cells (medium).

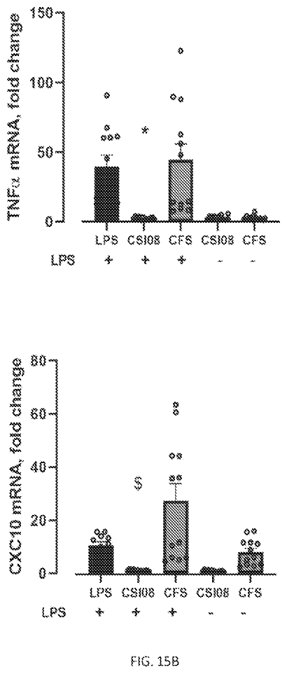

FIG. 14 shows adhesion of vegetative cells and spores B. clausii CSI08 to the HT-29-MTX. B. clausii CSI08 compared with L. fermentum.

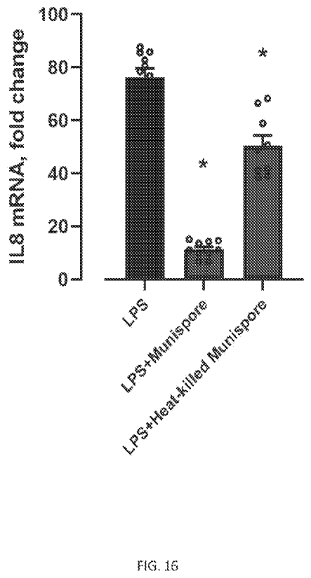

FIGS. 15A and 15B show the modulation of LPS-induced pro-inflammatory response by B. clausii CSI08 in HT-29 cell line, and the pattern of gene expression after co-incubation of HT-29 cells with B. clausii CSI08 and its CFS in unstimulated conditions (LPS-).

FIG. 16 shows modulation of LPS-induced IL8 gene expression by viable and heat-killed B. clausii CSI08.

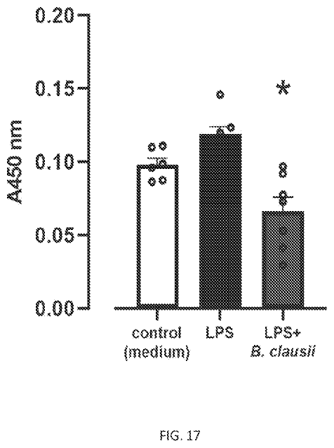

FIG. 17 shows NF-κB levels in the nuclear fractions of control HT-29s, cells exposed to LPS, and cells treated with B. clausii CSI08 prior to adding LPS.

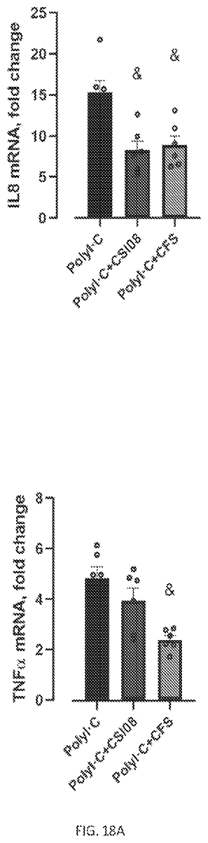

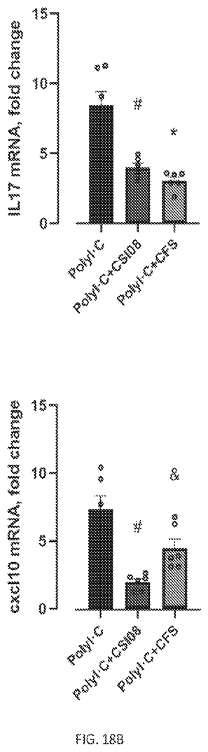

FIGS. 18A and 18B show modulation of PolyI·C-triggered pro-inflammatory response by B. clausii CSI08 in HT-29 cell line. qPCR analysis of IL-8, TNF-α, IL-17C, and CXCL10 gene expression 4 hours after exposure to PolyI·C in HT-29 cells preincubated with B. clausii CSI08 or its cell free supernatants (CFS).

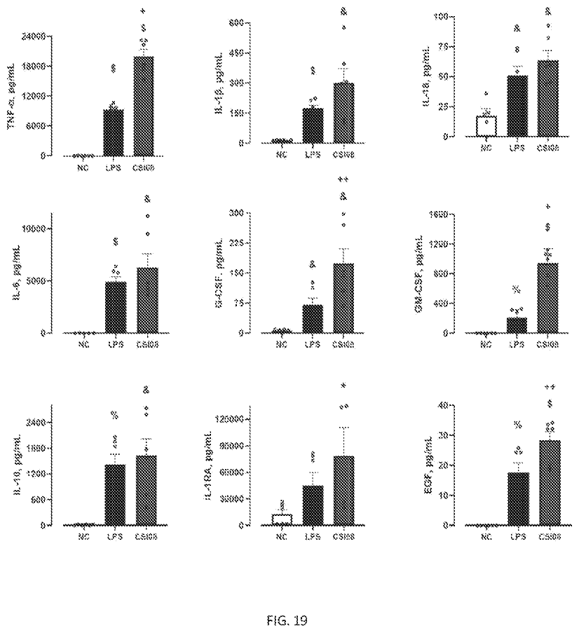

FIG. 19 shows immunostimulatory effect of CSI08. Cytokine levels in cell culture supernatants of U937-derived macrophages challenged by vegetative cells B. clausii CSI08 or LPS for 5 hours.

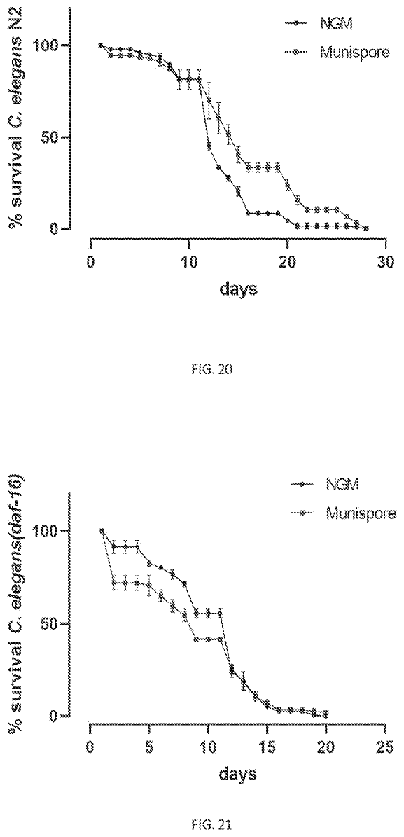

FIG. 20 shows the lifespan of C. elegans wild type N2 strain in control conditions (NGM) and lifespan of C. elegans wild type N2 fed with B. clausii CSI08.

FIG. 21 shows the lifespan of C. elegans daf-16 mutant worms in control conditions (NGM) and lifespan of C. elegans daf-16 mutants fed with B. clausii CSI08.

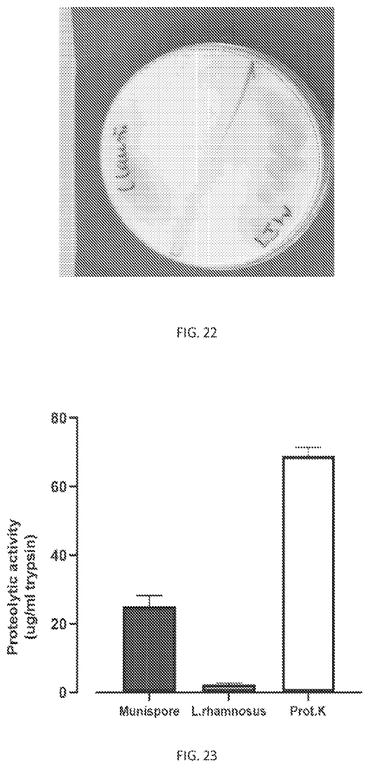

FIG. 22 shows B. clausii CSI08 is slightly caseolytic using streak method, as well as B. megaterium and B. clausii CSI08 caseolytic activity.

FIG. 23 shows proteolytic activity determined by EnzCheck® kit assay.

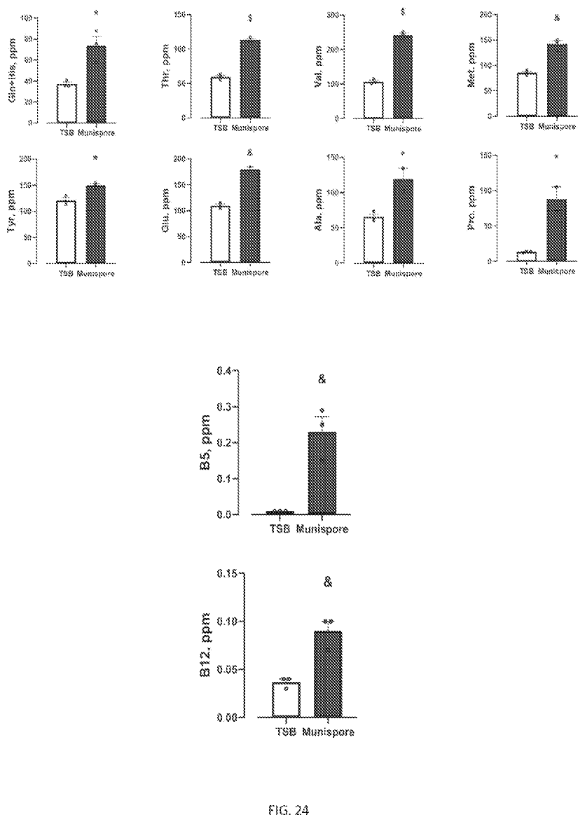

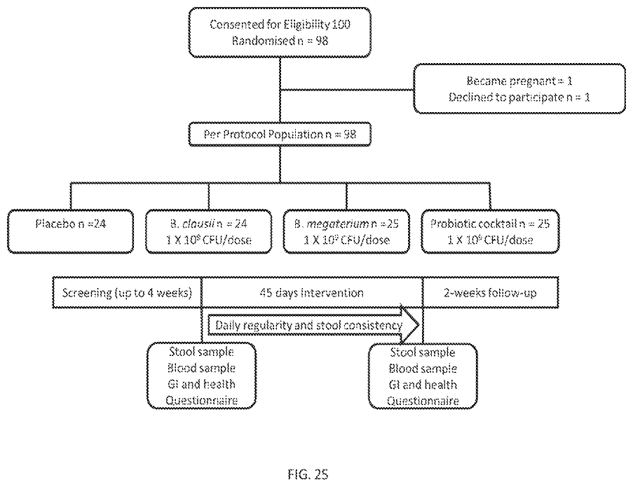

FIG. 24 shows concentrations of essential amino acids and vitamins in overnight cultures of B. clausii CSI08 determined by RP-HPLC-FLD and RP-HPLC-MS.

FIG. 25 shows a graphical flow chart of the study design.

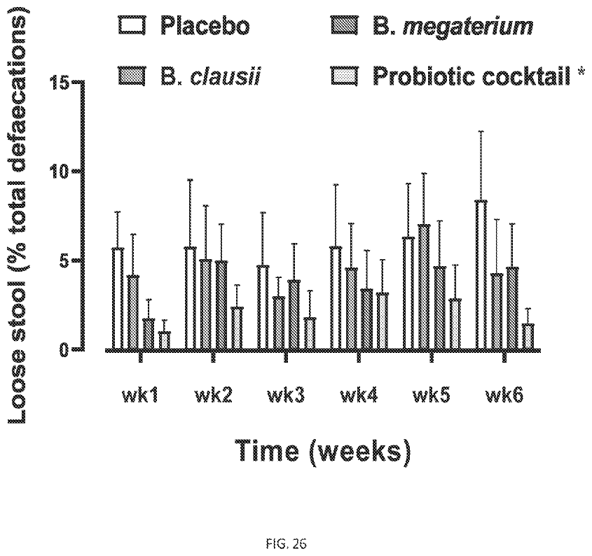

FIG. 26 shows the probiotic cocktail administered during the study significantly decreased the incidence of loose stool over the course of the study as compared to placebo control.

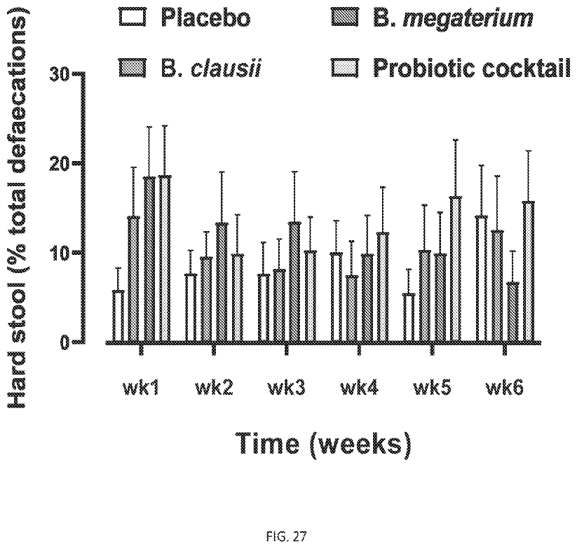

FIG. 27 shows no effect of any treatments administered during the study on percentage of hard stools as compared to placebo control.

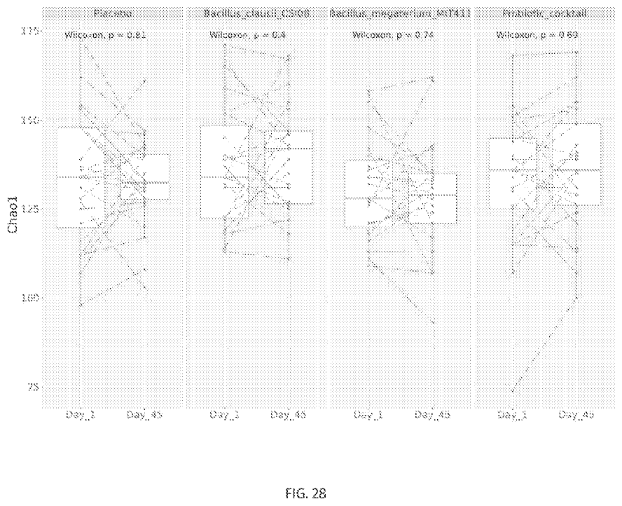

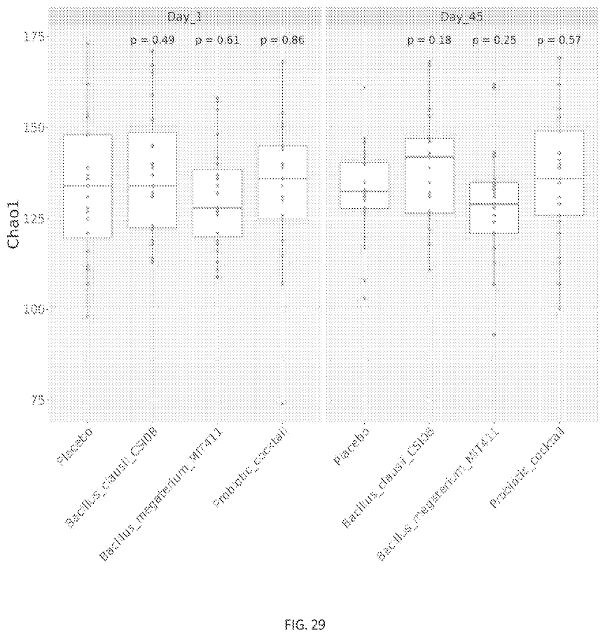

FIG. 28 is a boxplot showing the Chao1 values distribution in each experimental group for Day 1 and Day 45 of the study. Dotted lines connect the paired samples. A paired Wilcoxon test was used to compare the distribution of the groups.

FIG. 29 is a boxplot showing the Chao1 values distribution in each experimental group for Day 1 and Day 45 of the study. A Wilcoxon test was used to compare the distribution of each experimental group against the Placebo.

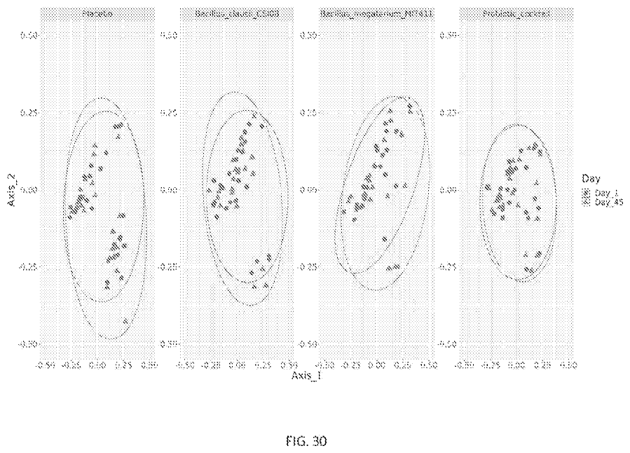

FIG. 30 illustrates PCoA clustering performed on the Bray-Curtis dissimilarity matrix.

### DETAILED DESCRIPTION OF THE INVENTION

### Genotypic Identification

The Applicant collaborated with Cornell University (Ithaca NY, USA) for genomic sequencing and identification.

### WGS DNA Composition

The whole genome sequence (WGS) was carried out by Cornell University.

The whole genome sequence was obtained for the Bacillus clausii isolate, assembled, and annotated by Cornell University. Bioinformatics analysis was completed at Cornell University and at Deerland Probiotics and Enzymes (Kennesaw GA, USA). DNA nucleotide content, base pair lengths for Bacillus clausii CSI08 genome, and marker sequences are shown below (Table 1). The genome size (4.2 Mbp) and % GC (44.6%) of Bacillus clausii CSI08 is consistent with that of a previously sequenced Bacillus clausii strain (4.3 Mbp and 44.6%, respectively) (https://www.ncbi.nlm.nih.gov/genome/?term=txid66692 [Organism:noexp]).

TABLE 1
Whole genome sequencing metrics of CSI08

| | | Number | Total | | | Average |
|---|---|---|---|---|---|---|
| | | of | length | | | coverage |
| Strain | Attempt | contigs | (nt) | GC (%) | N50 (nt) | (x) |
| Bacillus | 1 | 52 | 4,264,753 | 44.66 | 281,140 | 41.0102 |
| clausii | | | | | | |
| CSI08 | | | | | | |

gyrB Gene

Identifying gyrB gene polymorphism was carried out by the Applicant. The gyrB gene encodes DNA gyrase subunit B. DNA gyrase negatively supercoils closed circular double-stranded DNA in an ATP-dependent manner to maintain chromosomes in an underwound state.

Gene sequencing analysis using the gyrB gene polymorphism, a well-established method for species level discrimination of prokaryotes (Bavykin et al., 2004; Wang et al., 2007) showed that Bacillus clausii CSI08 was most closely related (>99%) to the Bacillus clausii group (Table 2).

TABLE 2
Distance matrix of gyrB gene

| Bacillus clausii subsp. | B106 | UBBC-07 |
|---|---|---|
| CSI08 | 99.983 | 98.952 |

The representative genomes were previously reviewed, curated by NCBI, and coordinated with the UniProt Consortium (NCBI, 2016; UniProt, 2016). R package SequinR coupled with the UniProt Consortium analysis was used to compare whole genome sequences (WGS) and GyrB sequence of Bacillus clausii CSI08 and two reference sequences (Table 3) for base pair length and GC content. Independent whole genome sequence (WGS) analysis by Deerland Probiotics and Enzymes identified CSI08 with a homology most similar to Bacillus clausii B106.

TABLE 3
Whole genome sequence comparison

| Bacillus clausii | | | Sequence |
|---|---|---|---|
| subsp. | Accession No. | % G/C | Length |
| CSI08 | JABBNL000000000.1 | 44.66% | 4,264,753 |
| B106 | GCA_002266625.1 | 44.63% | 4,253,129 |
| UBBC-07 | GCF_000981315.1 | 44.63% | 4,197,324 |

16S rRNA

Whole genome sequencing and 16S rRNA analysis for the presently claimed strain (Bacillus clausii CSI08), as compared to the two reference strains, exhibited an average nucleotide identity (ANI) score for 16S rRNA of >99% when compared to the B. clausii strain B106 (the genome sequence of Bacillus clausii B106 has been deposited in GenBank under the accession number. NFZO00000000). The genome size (4.2 MBP) and GC content (44.6%) for CSI08 was comparable to the two reference strains.

### FURTHER DEPOSITS AND ACCESSION NUMBERS

The whole genome sequence of Bacillus clausii UBBC07 has been deposited at DDBJ/ENA/GenBank under the accession no. LATY00000000.

Genome sequence data of Bacillus clausii strain CSI08 (Munispore) was deposited into NCBI GenBank database, and the genome sequence was annotated with the NCBI Prokaryotic Genome Annotation Pipeline (PGAP). The genome is publicly available, with GenBank Accession Number JABBNL000000000.1 for the strain, and available for instance at the link: Alkalihalobacillus clausii strain CSI08, whole genome shotgun sequenci-Nucleotide-NCBI (nih.gov).

Genome sequence data of Bacillus megaterium strain MIT411 (Renuspore) was deposited into NCBI GenBank database, and the genome sequence was annotated with the NCBI Prokaryotic Genome Annotation Pipeline (PGAP). The genome is publicly available, with GenBank Accession Number JABBNK000000000.1 for the strain, and available for instance at the link: Priestia megaterium strain MIT411, whole genome shotgun sequencing pro—Nucleotide—NCBI (nih.gov).

Genome sequence data of Bacillus coagulans strain CGI314 (Fortispore) was deposited into NCBI GenBank database, and the genome sequence was annotated with the NCBI Prokaryotic Genome Annotation Pipeline (PGAP). The genome is publicly available, with GenBank Accession Number JABBFU000000000.1 for the strain, and available for instance at the link: https://www.ncbi.nlm.nih.gov/nuccore/JABBFU000000000.1.

### Phylogenetic Placement Deerland Probiotics and Enzymes, Inc.

Genome-to-genome distance calculation (GGDC), a digital gold standard, is as reliable as DNA-DNA hybridization (DDH) (Auch et al., 2010). GGDC holds more discriminatory power for subspecies delineation and subsequently, was used as a confirmation of multiple alignment and phylogenetic analyses. GGDC yielded three calculation-based models that further verified Bacillus clausii CSI08 is a close relative to Bacillus clausii B106 (Table 3).

Although the conserved 16S rRNA sequence is a well-established method to compare and study phylogenies in bacteria, the high percentage of sequence similarity between closely related species limits its usefulness (Wang et al., 2007). High rates of 16S rRNA sequence similarity in closely related bacterial species are due to a slower rate of molecular evolution. Past research (Bavykin et al., 2004; Wang et al., 2007) supports the validity of using gyrB sequences as taxonomic biomarkers due to their rate of base substitutions and significant and reliable correlation with DNA-DNA Hybridization analysis (Dauga et al., 2002; Kasai et al., 1998; Wang et al., 2007). The gyrB encodes DNA gyrase B, and type II topoisomerase that plays an important role in DNA replication. Gyrase B subunits are encoded by the gyrB gene.

Phylogenetic analysis using the neighbor-joining (NJ) method (Saitou & Nei, 1987) placed Bacillus clausii CSI08 in a clade with Bacillus clausii B106 (FIG. 2). This confirms all previous genomic identity determinations. Bacillus clausii B106 has been placed in the Bacillus clausii group.

### Definitions

By “excipient” is meant any non-active ingredient that is added to form part of the final formulation.

By “probiotic” is meant a viable microbial supplement, which has a beneficial influence on a subject through its effects in the intestinal tract, urinary tract, vaginal tract, and/or other areas of a subject's body.

A “prebiotic” is used herein as a substrate, which has a beneficial effect on a probiotic and thus on a subject taking (e.g. administered) the probiotic. Suitable prebiotics may be selected from an inulin, an oligosaccharide, and/or a vitamin.

A “subject” as used herein includes a person suffering from any clinical condition related to a microbial imbalance as well as a person using bacterial preparations prophylactically, for wellness, or any other purpose including for instance benefitting from the administration of Bacillus clausii strain of this invention (e.g. CSI08). Optionally, the subject is a human, a patient, and/or a mammal.

By a “symbiotic product” is meant a combination of probiotic and prebiotic, which is synergy, have a beneficial influence on the patient.

By “hardy growth” is meant that bacteria show excellent growth.

The abbreviation “CFU” means colony forming units.

The present invention relating to a probiotic Bacilli strain capable of regenerating the in vivo flora in subjects will become apparent in the progress of the following detailed description.

According to a first aspect, the present invention comprises Bacillus clausii strain CSI08 alone or in combination with other probiotic Bacilli strains with essentially the same properties. Such other probiotic Bacilli strains include, but are not limited to a Bacillus coagulans strain and a Bacillus megaterium strain. Such other Bacilli strains further include a Bacillus coagulans strain and a Bacillus megaterium strain each filed today under these respective titles-their contents are incorporated herein in their entirety.

SEQ ID NO: 1, as recited in the claims attached hereto, comprises gyrB of Bacillus clausii CSI08.

SEQ ID NO: 2, as recited in the claims attached hereto, comprises 16S rRNA of Bacillus clausii CSI08.

SEQ ID NO: 3, as recited in the claims attached hereto, comprises the assembled whole genome sequence of Bacillus clausii CSI08.

The Bacillus clausii CSI08 strain claimed herein, with reference to at least 97% identity to SEQ ID NO: 1 and/or 2; or to at least 97% identity to SEQ ID NO: 3, has the following properties:

In order to determine the genus and species of the strains disclosed herein, the whole genome was sequenced. The amount and composition of the strains were identified and determined.

The strain was shown to possess little to no antibiotic resistance and no safety concerns.

The strain was found to show stability toward acid and bile and showed heat tolerance. The strain produced a natural antibiotic substance in the form of bacteriocins.

According to a second aspect, the Bacilli strain of the present invention is suitable for medical use in preventing or treating vaginal infections, urinary tract infections and gastrointestinal diseases, as well as, improving immune health, protection against oxidative stress, cleansing and detoxification, metabolic health and cardiovascular health.

In another preferred embodiment, a composition such as a pharmaceutical composition is provided comprising Bacillus clausii CSI08 alone or in combination with other probiotic Bacilli strains with similar and/or essentially the same properties, together with a pharmaceutically acceptable carrier and/or diluent. Such other probiotic Bacilli strains include, but are not limited to a Bacillus coagulans strain and a Bacillus megaterium strain. The bacterial strains are formulated into compositions such as pharmaceutical formulations in order to allow the easy administration of the probiotic strains and by means known to the man skilled in the art.

Bacillus coagulans has been proven able to alleviate symptoms of irritable bowel syndrome (Sudha et al., 2018), improve muscle integrity and cytokine response (Gepner et al., 2017; Jager et al., 2018), modulate the gut microbiome and the immune response (Kimmel et al., 2010), reduce function intestinal gas symptoms (Kalman et al., 2009), reduce the instance and duration of diarrhea (Dolin et al., 2009), improve the symptoms of functional abdominal pain and bloating (Hun et al., 2009), protect against acetaminophen induced acute liver injury (Neag et al., 2020), enhance butyrogenesis (Sasaki et al., 2020), reduce severity of bacterial vaginosis (Sudha et al., 2012), and reduce cholesterol (Sudha et al., 2012) all in vivo. Bacillus coagulans has also shown to induce immune response and anti-inflammatory action (Jensen et al., 2017), improve plant protein digestion (Keller et al., 2017), adhere to Caco-2 cells (Sharma & Kanwar, 2017), improve colonic microenvironment in patients with ulcerative colitis (Sasaki et al., 2020), reduce the adhesion, cytotoxicity and induction of apoptosis caused by S. typhimurium in HT-29 cells (Kawarizadeh et al., 2019), hydrolyze lactose from whey protein (Liu et al., 2019), and enhancing t-cell response (Baron, 2009) all in vitro.

Bacillus clausii has been proven efficacious in preventing recurrent respiratory infections (Marseglia et al., 2007), reducing duration and severity of diarrhoea (Sudha et al., 2019) in vivo. Bacillus clausii has also been proven capable to produce protein hydrolysates with antimicrobial and antioxidant capacity (Rochin-Medina et al., 2017), protect against acetaminophen induced acute liver injury (Neag et al., 2020), inhibit cytotoxic effects induced by Clostridium difficile and Bacillus cereus toxins (Ripert et al., 2016) in vitro.

Bacillus megaterium has been shown to exert protective effects against oxidative stress both in vitro and in vivo (Mazzoli et al., 2019). Bacillus megaterium has also been shown capable of adapting and surviving in acid stress conditions and chelating heavy metals in vitro (Ferreira et al., 2019).

In an embodiment, the probiotic bacteria employed in this invention are used in bacterial concentration of 10⁶-10¹³ CFU (colony forming units), for instance as a daily dose, including any amount or range that is included in said range. In an embodiment, the bacteria are employed in an amount of 10⁷-10¹² CFU, or 10⁸-10¹¹ CFU, or 10⁹-10¹⁰ CFU, or for instance in an amount of about 10⁶, about 10⁷, about 10⁸, about 10⁹, about 10¹⁰, about 10¹¹, about 10¹², and/or about 10¹³ CFU, and any amount or range including or between said amounts. In an embodiment, a composition of this invention comprises, consists essentially of, consists of, and/or is characterized by about 10⁶-about 10¹³ CFU such as about 10⁹ Bacillus clausii CSI08. In an embodiment, a composition of this invention comprises Bacillus clausii CSI08 (for instance about 10⁹ CFU) in combination with Bacillus megaterium MIT411 and/or Bacillus coagulans CGI314. In an embodiment, a composition of this invention is orally administered in capsule form. In an embodiment, Bacillus clausii CSI08 is in spore form, or is not in spore form.

In certain embodiments, compositions comprising Bacillus clausii CSI08 can include one or more dry carriers selected from the group consisting of trehalose, maltodextrin, rice flour, microcrystalline cellulose, stearate, magnesium inositol, fructooligosaccharide, galactooligosaccharide, dextrose, dried dairy products, and the like. In certain embodiments, the dry carrier can be added to the compositions comprising Bacillus clausii CSI08 in a weight percentage of from about 1% to about 95% by weight of the composition.

In certain embodiments, the compositions comprising Bacillus clausii CSI08 can include one or more liquid or gel-based carriers, selected from the group consisting of water and physiological salt solutions, urea, alcohols and derivatives thereof (e.g., methanol, ethanol, propanol, butanol), glycols (e.g., ethylene glycol, propylene glycol), and the like; natural or synthetic flavorings and food-quality coloring agents, all compatible with the organism; thickening agents selected from the group consisting of corn starch, guar gum, xanthan gum, and the like; one or more spore germination inhibitors selected from the group consisting of hyper-saline carriers, methylparaben, guargum, polysorbate, preservatives, and the like. In certain embodiments, the one or more liquid or gel-based carrier(s) can be added to the compositions comprising Bacillus clausii CSI08 in a weight/volume percentage of from about 0.6% to about 95% weight/volume of the composition. In certain embodiments, the natural or synthetic flavoring(s) can be added to the compositions comprising Bacillus clausii CSI08 in a weight/volume percentage of from about 3.0% to about 10.0% weight/volume of the composition. In certain embodiments, the coloring agent(s) can be added to the compositions comprising Bacillus clausii CSI08 in a weight/volume percentage of from about 1.0% to about 10.0% weight/volume of the composition. In certain embodiments, the thickening agent(s) can be added to the compositions comprising Bacillus clausii CSI08 in a weight/volume percentage of about 2% weight/volume of the composition. In certain embodiments, the one or more spore germination inhibitors can be added to the compositions comprising Bacillus clausii CSI08 in a weight/volume percentage of about 1% weight/volume of the composition.

### Delivery System

Suitable dosage forms include tablets, capsules, solutions, suspensions, powders, gums, and confectionaries. Sublingual delivery systems include, but are not limited to, dissolvable tabs under and on the tongue, liquid drops, and beverages. Edible films, hydrophilic polymers, oral dissolvable films, or oral dissolvable strips can be used. Other useful delivery systems comprise oral or nasal sprays or inhalers, and the like. Suitable dosage forms include tablets, capsules, solutions, suspensions, powders, gums, and confectionaries. Sublingual delivery systems include, but are not limited to, dissolvable tabs under and on the tongue, liquid drops, and beverages. Edible films, hydrophilic polymers, oral dissolvable films, or oral dissolvable strips can be used. Other useful delivery systems comprise oral or nasal sprays or inhalers, and the like.

For oral administration, probiotics may be further combined with one or more solid inactive ingredients for the preparation of tablets, capsules, pills, powders, granules, or other suitable dosage forms. For example, the active agent may be combined with at least one excipient selected from the group consisting of fillers, binders, humectants, disintegrating agents, solution retarders, absorption accelerators, wetting agents, absorbents, and lubricating agents. Other useful excipients include, but are not limited to, magnesium stearate, calcium stearate, mannitol, xylitol, sweeteners, starch, carboxymethylcellulose, microcrystalline cellulose, silica, gelatin, silicon dioxide, and the like

In certain embodiments, the components of compositions administered according to the methods of the present disclosure, together with one or more conventional adjuvants, carriers, or diluents, may thus be placed into the form of pharmaceutical compositions and unit dosages thereof. Such forms include: solids, and in particular, tablets, filled capsules, powder and pellet forms; liquids, and in particular, aqueous or non-aqueous solutions, suspensions, emulsions, elixirs; and capsules filled with the same; all for oral use, suppositories for rectal administration, and sterile injectable solutions for parenteral use. Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed.

The components of the compositions administered according to the methods of the present disclosure can be administered in a wide variety of oral and parenteral dosage forms. It will be obvious to those skilled in the art that the following dosage forms may comprise, in certain embodiments, as the active component, either a chemical compound of the present disclosure or a pharmaceutically acceptable salt of a chemical compound of the present disclosure.

For preparing pharmaceutical compositions to be administered according to the methods of the present disclosure, pharmaceutically acceptable carriers can be either solid or liquid. Solid form preparations include powders, tablets, pills, capsules, cachets, suppositories, and dispersible granules. A solid carrier can be one or more substances that may also act as diluents, flavoring agents, solubilizers, lubricants, suspending agents, binders, preservatives, tablet disintegrating agents, or encapsulating materials.

In powders, the carrier is a finely divided solid, which is in a mixture with the finely divided active component. In tablets, the active component is mixed with the carrier having the necessary binding capacity in suitable proportions and compacted in the shape and size desired.

In certain embodiments, powders and tablets administered according to methods of the present disclosure preferably may contain from five or ten to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier providing a capsule in which the active component, with or without additional carriers, is surrounded by a carrier, which is thus in association with it. Similarly, cachets and lozenges are included. Tablets, powders, capsules, pills, cachets, and lozenges are included. Tablets, powders, capsules, pills, cachets, and lozenges can be used as solid forms suitable for oral administration.

Liquid preparations include, but are not limited to, solutions, suspensions, and emulsions, for example, water or water-propylene glycol solutions. For example, parenteral injection liquid preparations can be formulated as solutions in aqueous polyethylene glycol solution. In certain embodiments, chemical compounds administered according to methods of the present disclosure may thus be formulated for parenteral administration (e.g., by injection, for example, bolus injection or continuous infusion) and may be presented in unit dose for administration in ampoules, pre-filled syringes, small-volume infusion, or in multi-dose containers with an added preservative. The compositions may take such forms as suspensions, solutions, or emulsions in oily or aqueous vehicles, and may contain formulation agents such as suspending, stabilizing, and/or dispersing agents. Alternatively, the active ingredient may be in powder form, obtained by aseptic isolation of sterile solid or by lyophilization from solution, for constitution with a suitable vehicle, e.g., sterile, pyrogen-free water, before use.

Aqueous solutions suitable for oral use can be prepared by dissolving the active component in water and adding suitable colorants, flavors, stabilizing and thickening agents, as desired. Aqueous suspensions suitable for oral use can be made by dispersing the finely divided active component in water with viscous material, such as natural or synthetic gums, resins, methylcellulose, sodium carboxymethylcellulose, or other well-known suspending agents.

Compositions suitable for topical administration in the mouth include, but are not limited to: lozenges comprising the active agent in a flavored base, usually sucrose and acacia or tragacanth; pastilles comprising the active ingredient in an inert base such as gelatin and glycerine or sucrose and acacia; and mouthwashes comprising the active ingredient in suitable liquid carrier.

Solutions or suspensions are applied directly to the nasal cavity by conventional means, for example, with a dropper, pipette, or spray. The compositions may be provided in single or multi-dose form. In compositions intended for administration to the respiratory tract, including intranasal compositions, the compound will generally have a small particle size, for example, of the order of 5 microns or less. Such a particle size may be obtained by means known in the art, for example, by micronization.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packaged tablets, capsules, and powders in vials or ampoules. Also, the unit dosage form can be a capsule, tablet, cachet, or lozenge itself; or it can be the appropriate number of any of these in packaged form.

Tablets, capsules, and lozenges for oral administration and liquids for oral use are preferred compositions. Solutions or suspensions for application to the nasal cavity or to the respiratory tract are preferred compositions. Transdermal patches for topical administration to the epidermis are preferred.

Further details on techniques for formulation and administration may be found in the latest edition of REMINGTON'S PHARMACEUTICAL SCIENCES (Mack Publishing Co., Easton, PA).

In certain embodiments, compositions of the present invention including compositions administered according to the methods of the present disclosure may also include one or more excipients, most preferably one or more nutraceutical or pharmaceutical excipients. Compositions containing one or more excipients and incorporating one or more probiotics can be prepared by procedures known in the art. Optionally, compositions can include one or more adjuvants, excipients, carriers, buffers, diluents, and/or other customary pharmaceutical auxiliaries. For example, probiotics can be formulated into tablets, capsules, powders, suspensions, solutions for oral administration, solutions for parenteral administration including intravenous, intradermal, intramuscular, and subcutaneous administration, and solutions for application onto patches for transdermal application with common and conventional barriers, binders, diluents, and excipients.

In certain embodiments, nutraceutical compositions including nutraceutical compositions administered according to the methods of the present disclosure may include and may be administered in combination with a pharmaceutically acceptable carrier. In certain embodiments, the active ingredients in such formulations may comprise from about 1% by weight to about 99% by weight. In other embodiments, the active ingredients in such formulations may comprise from about 0.1% by weight to about 99.9% by weight. “Pharmaceutically acceptable carrier” means any carrier, diluent, or excipient that is compatible with the other ingredients of the formulation and not deleterious to the user. Useful excipients include, but are not limited to, microcrystalline cellulose, magnesium stearate, calcium stearate, any acceptable sugar (e.g., mannitol, xylitol), and the like, and for cosmetic use, a water or an oil base may be used, or mixture thereof including such as an emulsion.

### Routes of Administration

The strain Bacillus clausii CSI08 or a composition comprising a strain of the present invention may be administered by any route, including, but not limited to, oral, sublingual, buccal, ocular, pulmonary, rectal, vaginal, urethral, ureteral, and parenteral administration, or as an oral or nasal spray (e.g., inhalation of nebulized vapors, droplets, or solid particles). Parenteral administration includes, for example, intravenous, intramuscular, intraarterial, intraperitoneal, intranasal, intravaginal, intravesical (e.g., to the bladder), intradermal, transdermal, topical, or subcutaneous administration. Also contemplated within the scope of the invention is the instillation of a pharmaceutical composition in the body of the patient in a controlled formulation, with systemic or local release of the drug to occur at a later time. For example, the drug may be localized in a depot for controlled release to the circulation, or for release to a local site.

Pharmaceutical compositions of the invention may be those suitable for, and formulated for, any of the routes identified above, including for instance oral, rectal, bronchial, nasal, pulmonal, topical (including buccal and sub-lingual), transdermal, vaginal, urethral, ureteral, or parenteral (including cutaneous, subcutaneous, intramuscular, intraperitoneal, intravenous, intraarterial, intracerebral, intraocular injection, or infusion) administration, or those in a form suitable for administration by inhalation or insufflation, including powders and liquid aerosol administration, or by sustained release systems. Suitable examples of sustained release systems include semipermeable matrices of solid hydrophobic polymers containing the compound of the invention, which matrices may be in the form of shaped articles, e.g., films or microcapsules.

The embodiments described above may be further understood in connection with the following Examples. In addition, the following non-limiting examples are provided to illustrate the invention. However, the person skilled in the art will appreciate that it may be necessary to vary the procedures for any given embodiment of the invention, e.g., vary the order or steps.

### EXAMPLES

### Example 1

### Characterisation of Bacillus clausii Strain CSI08

### Temperature Stability:

Bacillus clausii CSI08 spores in PBS (pH 7.72) are stable at 45° C., and 75° C. from 30 seconds to 3 minutes, with some reduction in spore counts after treatment at 90° C. for 3 minutes.

FIG. 4 shows the stability of MuniSpore in PBS during a pasteurization process. Data represent the mean±SEM. Spore counts compared to 0 min. *P<0.0001.

Bacillus clausii CSI08 spores are stable in a pasteurization process and during other manufacturing methodologies used in food & beverage.

The strain shows bile stability and the strain shows acid stability. The survivability of B. clausii CSI08 spores was determined in acidic and bile salt conditions in nutrient broth media for 24 hours. Total spore count for B. clausii CSI08 does not show a significant reduction in viability or concentration after contact with acidic conditions or bile salt concentrated nutrient broth for 3 hours. At the most extreme conditions, pH 1.2 and 0.45% bile salt concentration a significant decrease in counts was observed after 24 hours. Both pH and bile salts at these concentrations and exposures greatly exceed the conditions in the stomach and intestinal tract.

FIG. 5 shows pH Survivability of B. clausii CSI08 spores at 0-, 3-, and 24-hour timepoints.

FIG. 6 shows bile salt survivability of B. clausii CSI08 spores at 0-, 3-, and 24-hour timepoints.

B. clausii CSI08 spores are not sensitive to acid conditions above 1.2 for 24 h and are resistant to bile salts concentrations up to 0.30% for 24 h. Therefore, B. clausii CSI08 spores can survive in low pH food and beverages (e.g., fruit juices) and in the stomach and intestine conditions.

The strain shows heat tolerance. Bacillus clausii CSI08 spores in PBS (pH 7.72) are stable at 45° C., and 75° C. from 30 seconds to 3 minutes, with some reduction in spore counts after treatment at 90° C. for 3 min.

Bacillus clausii CSI08 spores are stable in a pasteurization process and during other manufacturing methodologies in food & beverage.

Survivability of B. clausii CSI08 spores during an in vitro simulated digestion process (gastric phase: 0.3% pepsin pH=3 for 2 h; small intestinal phase: 0.1% pancreatin, 0.3% bile salts pH 7.5 for 2 h) was investigated. No reduction in B. clausii CSI08 spores counts after exposure to simplified gastric and small intestinal conditions detected. There is a decrease in L. rhamnosus counts at the small intestinal phase.

Results show the ability of Bacillus clausii CSI08 spores to efficiently survive the transit through the upper digestive tract.

FIG. 7 shows survival of Bacillus clausii CSI08 spores and L. rhamnosus GG under gastric and small intestinal digestion conditions simulated in vitro. Data represent the mean±SEM. Survival compared to 0 h *P<0.0001.

The strain produces a natural antibiotic substance in the form of bacteriocins.

The vegetative form B. clausii CSI08 is active against the common intestinal pathogens E. coli and Salmonella having potential to protect and restore gut microbial communities.

The vegetative form B. clausii CSI08 has also proven to have antimicrobial activity against the well-known skin and urinary tract pathogen Pseudomonas aeruginosa in liquid media.

TABLE 4
Antimicrobial activity

| | Inhibition capacity | | |
|---|---|---|---|
| Conditions | E. coli | S. enteritidis | P. aeruginosa |
| Solid media | + | + | − |
| Liquid media | + | + | + |

Table 4. Bacillus clausii CSI08 spores show antimicrobial activity against common pathogens. Antimicrobial activity detected (+), no antimicrobial activity observed (−).

The vegetative form B. clausii CSI08 revealed antimicrobial activity against E. coli, Salmonella, S. aureus and Pseudomonas aeruginosa in liquid medium.

FIGS. 8 and 9 show antimicrobial activity of Munispore (B. clausii CSI08) in liquid culture. Values represent average concentration lof₁₀ CFU/ml±standard deviation. *p<0.05, ****p<0.0001.

Therefore, the vegetative form B. clausii CSI08 has the potential to crowd out bacterial pathogens and maintain a healthy gut and skin microbiome.

Antioxidant activity: The total antioxidant activity of the vegetative form B. clausii CSI08 was compared with L. rhamnosus GG (DI_AS_030).

Munispore has higher amount of antioxidant activity than L. rhamnosus GG.

FIG. 10 shows total antioxidant capacity of B. clausii CSI08 and L. rhamnosus GG. Results show average concentration of Trolox equivalents in nmole/g (n=3)±standard error.

The increased levels of Trolox equivalent concentration in Bacillus clausii CSI08 spores can neutralize the effects of reactive oxidative species. The antioxidant properties in Bacillus clausii CSI08 spores enables the strain to act as producer of antioxidant enzymes and molecules that results in alleviation of oxidative damage.

Therefore, Bacillus clausii CSI08 spores are a potential probiotic with strong antioxidant properties

The antioxidant activity of the vegetative form B. clausii CSI08 was confirmed using a cell culture model and an in vivo C. elegans model of oxidative stress.

The vegetative form B. clausii CSI08 attenuates hydrogen peroxide-induced reduction in cell viability of epithelial cells (HT-29 cell line).

The vegetative form B. clausii CSI08 attenuates hydrogen peroxide-induced decrease in survival rate of C. elegans N2 after oxidative stress.

FIG. 11 shows the cytoprotective effect of the vegetative form B. clausii CSI08 on H₂O₂-exposed epithelium. Values are the means±SEM.*−P<0.0001 vs H₂O₂

FIG. 12 shows the survival rate of C. elegans N2 fed with 10⁸ and 10⁹ CFU/ml B. clausii CSI08, followed by oxidative stress caused by H₂O₂. NGM-control-fed nematodes; Vit C-positive control. Values are the average of two independent experiments (n=100/condition). *p<0.05 vs NGM (+H₂O₂).

Bacillus clausii CSI08 spores demonstrate a reversal of oxidative stress in mammalian cell lines and a host organism.

Adhesion ability and cytotoxic effect: the vegetative form B. clausii CSI08 doesn't influence the viability of the intestinal epithelial cells (HT-29 model).

The vegetative form and spores of B. clausii CSI08 have the significant ability to adhere to the mucous-producing cell line HT-29-MTX.

Adhesion of the vegetative form and spores of B. clausii CSI08 to the non-mucous producing intestinal epithelial cell line (HT-29) is negligible, graph not shown.

FIG. 13 shows the effect of vegetative cells B. clausii CSI08 on HT-29 cell viability. B. clausii CSI08 compared with untreated cells (medium). Values are means±SEM.

FIG. 14 shows adhesion of vegetative cells and spores B. clausii CSI08 to the HT-29-MTX. B. clausii CSI08 compared with L. fermentum. Values are means±SEM

Bacillus clausii CSI08 does not negatively impact mammalian cell viability and can adhere to the gut lining.

Anti-inflammatory activity: the vegetative form of B. clausii CSI08 and its cell-free supernatants ability to attenuate LPS-triggered pro-inflammatory response was investigated in an in vitro model of intestinal epithelium (HT-29 cell line).

Pre-treatment with the vegetative form of B. clausii CSI08, but not its supernatants (CFS), result in strong suppression of the expression of genes connected to pro-inflammatory response after exposure HT-29 cells to LPS.

FIGS. 15A and 15B show the modulation of LPS-induced pro-inflammatory response by B. clausii CSI08 in HT-29 cell line. Also shown the pattern of gene expression after co-incubation of HT-29 cells with B. clausii CSI08 and its CFS in unstimulated conditions (LPS-). $ p<0.05 vs LPS, #p<0.001 vs LPS, *p<0.0001 vs. LPS.

The vegetative form of B. clausii CSI08, but not its cell free supernatants, can reduce pro-inflammatory response in HT-29 cells triggered by LPS.

B. clausii CSI08 partially retains the ability to suppress pro-inflammatory response triggered by LPS in HT-29 cells after heat-inactivation.

FIG. 16 shows modulation of LPS-induced IL8 gene expression by viable and heat-killed B. clausii CSI08. *p<0.0001.

B. clausii CSI08 can down regulate LPS-induced NF-κB activation in HT-29 cell line exposed to LPS.

FIG. 17 shows NF-κB levels in the nuclear fractions of control HT-29s, cells exposed to LPS, and cells treated with B. clausii CSI08 prior to adding LPS. LPS versus LPS+B. clausii*p<0.001 according to t-test.

B. clausii CSI08's immunomodulatory efficacy is mediated in part via Nf-κB pathway.

The vegetative form B. clausii CSI08 and its cell-free supernatants ability to attenuate PolyI·C-triggered pro-inflammatory response (a viral mimetic) was investigated in an in vitro model of intestinal epithelium (HT-29 cell line).

Pre-treatment with CSI08 and its supernatants (CFS) result in substantial suppression of the expression of genes connected to pro-inflammatory response after exposure HT-29 cells to PolyI·C.

FIGS. 18A and 18B show modulation of PolyI·C-triggered pro-inflammatory response by B. clausii CSI08 in HT-29 cell line. qPCR analysis of IL-8, TNF-α, IL-17C, and CXCL10 gene expression 4 h after exposure to PolyI·C in HT-29 cells preincubated with B. clausii CSI08 or its cell free supernatants (CFS). $ p<0.05, & p<0.01; #p<0.001, *p<0.0001.

The vegetative form of B. clausii CSI08, and its cell free supernatants, can reduce pro-inflammatory response in HT-29 cells triggered by PolyI·C, a viral mimetic.

Immune effect: B. clausii CSI08 can provoke the strong immune response in U937-derived macrophages

The effect of the vegetative form B. clausii CSI08 on the innate immune system was investigated using the cell model of U937-derived macrophages.

The vegetative form of B. clausii CSI08 stimulated a robust immune response, resulted in secretion of high levels of pro-inflammatory (TNF-α, IL-1β, IL-18), regulatory (G-CSF, GM-CSF, IL-6) and anti-inflammatory (IL-10, IL-IRA, EGF) cytokines by macrophages.

FIG. 19 shows immunostimulatory effect of CSI08. Cytokine levels in cell culture supernatants of U937-derived macrophages challenged by vegetative cells B. clausii CSI08 or LPS for 5 h. Values are the means±SEM. *p<0.05, & p<0.01, % p<0.001; $ p<0.0001 LPS or CSI08 vs NC (negative control). ++p<0.05; +p<0.0001 CSI08 vs LPS.

B. clausii CS108 prolongs the lifespan of C. elegans N2 worms compared with control condition-NGM medium. This effect is DAF-16-dependent.

FIG. 20 shows the lifespan of C. elegans wild type N2 strain in control conditions (NGM) and lifespan of C. elegans wild type N2 fed with B. clausii CSI08. The average of two independent assays (n=100/replicate/condition).

Transcription factor DAF-16 (orthologue of the FOXO) mediates the effect of B. clausii CSI08 on C. elegans lifespan.

The data indicates the role of B. clausii CSI08 as a potential anti-inflammatory effector or immune activator.

FIG. 21 shows the lifespan of C. elegans daf-16 mutant worms in control conditions (NGM) and lifespan of C. elegans daf-16 mutants fed with B. clausii CSI08. The average of two independent assays (n=100/replicate/condition).

B. clausii CSI08 demonstrates strong immunoprotective effects in a host organism.

B. clausii CSI08 exhibits moderate caseolytic/protease activity.

B. clausii CSI08 was weakly positive for casein degradation/Cascolytic activity on skim milk agar plates.

Quantitative analysis of B. clausii CSI08's caseolytic activity was evaluated using a commercial kit employing fluorescently tagged casein derivatives.

B. clausii CSI08 displays moderate protease activity (L. rhamnosus as a negative control).

FIG. 22 shows B. clausii CSI08 is slightly caseolytic using streak method. The figure shows B. megaterium and B. clausii CSI08 caseolytic activity.

FIG. 23 shows proteolytic activity determined by EnzCheck® kit assay. Data represent the mean±SEM.

B. clausii CSI08 exhibits moderate caseolytic/protease activity

B. clausii CSI08's diverse carbohydrate assimilation profile: B. clausii CSI08 was positive for 20 carbohydrates out of 49 tested, using commercial API 50 CH strip.

The majority of these carbohydrates were simple sugars such as D-Ribose, D-Glucose, D-Fructose, D-Mannose, and Disaccharides such as Trehalose, Sucrose and Cellobiose etc. Additionally, there were compounds belonging to cyanogenic glycoside (amygdalin), amine sugars (Glucosamine) and coumarin glucoside (Aesculin) that B. clausii CSI08 can metabolise. B. clausii CSI08 can also metabolise polysaccharide Glycogen and shows a weak ability to metabolise Amidon (Starch).

TABLE 5
Carbohydrates fermented by B. clausii CSI08

| | B. | | B. |
|---|---|---|---|
| | clausii | | clausii |
| Carbohydrate | CSI08 | Carbohydrate | CSI08 |
| GLYCEROL | + | N-ACETYLGLUCOSAMINE | + |
| 1-ARABINOSE | + | AMYGDALIN | (+) |
| D-RIBOSE | + | ESCULIN FERRIC CITRATE | + |
| D-GLUCOSE | + | D-CELLOBIOSE | + |
| D-FRUCTOSE | + | D-SACCHAROSE | + |
| D-MANNOSE | + | D-TREHALOSE | + |
| L-RHAMNOSE | + | D-RAFINOSE | (+) |
| DULCITOL | + | AMIDON | (+) |
| D-MANNITOL | + | GLYCOGEN | (+) |
| D-SORBITOL | + | D-TAGATOSE | + |

Table 5: List of Carbohydrates that are effectively fermented using API 50 Ch strips.

These data suggest that B. clausii CSI08 could help digest these compounds in the gut.

B. clausii CSI08 is Esteroloytic and has Phosphatase and β-Galactosidase Enzymes: B. clausii CSI08 was positive for esterase, phosphohydrolase, and β-galactosidase activity using API ZYM kit which implies that.
B. clausii CSI08 may generate free fatty acids from the action of esterase in the presence of an appropriate lipid source.
Beta-galactosidase-enzyme that catalyzes lactose hydrolysis into glucose and galactose and, importantly, is responsible for formation of galacto-oligosaccharides (GOS) promoting the growth of Bifidobacterium and Lactobacillus species
Phosphohydrolase catalyzes the hydrolysis of phosphates
No detrimental enzymes activity detected (β-glucuronidase, α-chymotrypsin and β-glucosaminidase).

TABLE 6
Enzymatic profile of B. clausii CSI08

| | | B. clausii |
|---|---|---|
| | | CSI08 |
| Esterase activity | Esterase (C4:0) | + |
| | Esterase (C8:0) | + |
| Lipase activity | Lipase (C14:0) | − |
| Peptidase activity | Leucine ary lamidase | − |
| | Valine ary lamidase | − |
| | Cystine ary lamidase | − |
| Proteinase activity | Trypsin | − |
| | α-chymotrypsin | − |
| Phosphatase activity | Acid phosphatase | − |
| | Alkaline phosphatase | − |
| | Phosphohydrolyase | + |
| Glycosidase activity | α-Galactosidase | − |
| | β-Galactosidase | + |
| | β-Glucuronidase | − |
| | α-Glucosidase | − |
| | β-Glucosidase | − |
| | β-Glucosaminidase | − |
| | α-Mannosidase | − |
| | α-Fucosidase | − |

Table 6: Enzymatic Profile of B. clausii CSI08 Using API ZYM Kit

This indicates the potential ability of B. clausii CSI08 to break down lactose and fats. These data suggest that B. clausii CSI08 could help digest these molecules in the gut.

Proteomic Analysis of B. clausii CSI08 Identifies Proteins with Potential Probiotic Benefits:

Extracellular secretions of B. clausii CSI08 grown in TSB broth for 24 h were sent to mass spectrometry to identify proteins released by the probiotic strain. A total of 29 proteins were detected of which 5 had potential probiotic benefits.

TABLE 7
Proteomic Analysis of B. clausii CSI08

| Proteins of interest | Potential roles |
|---|---|
| Chaperonin GroEL | Involved in protein folding and |
| | preservation |
| 2,3-butanediol | Involved in metabolism of branched |
| dehydrogenase | amino-acids - flavor properties. |
| Alpha-glucosidase/alpha- | Involved in digestion of carbohydrates |
| galactosidase | |
| Trypsin-like peptidase | Involved in digestion of proteins |
| domain-containing protein | |
| C40 family peptidase | Involved in digestion of proteins |

These data confirms previous in vitro results showing how B. clausii CSI08 can help in digestion of proteins and carbohydrates and can contribute to the production of branched amino-acids.

B. clausii CSI08 can produce essential amino acids and group B vitamins: The ability of B. clausii CSI08 to synthesize essential amino acids and vitamins during cultivation in liquid medium (TSB) was assessed. The results suggest the production of several amino acids (alanine, glutamine and glutamic acid, histidine, methionine, proline, tyrosine, and threonine) and two group B vitamins (Pantothenic acid/B5 and Cyanocobalamin/B12) by B. clausii CSI08.

FIG. 24 shows concentrations of essential amino acids and vitamins in overnight cultures of B. clausii CSI08 determined by RP-HPLC-FLD and RP-HPLC-MS correspondingly. Values are means±SEM. *p<0.05, & p<0.01, $ p<0.0001.

B. clausii CSI08 can produce essential amino acids and group B vitamins

### Example 2

### Assess Adhesion Ability to an In Vitro Model of Intestinal Epithelium

Cell lines: Human Colorectal Adenocarcinoma Cell Line HT-29 and mucous-secreting cell line HT-29-MTX were propagated using low glucose DMEM medium supplemented with 10% Fetal Bovine Serum, 2 mM glutamine, 100 U/ml penicillin, 100 μg/ml streptomycin, and 2 μg/ml amphotericin B in a 5% CO₂ atmosphere at 37° C.

Cells were seeded onto 24-well plates at a density 5×10⁵ cell/well and cultured for 21-28 days to complete maturation. Media was replaced every 2-3 days.

Prior to experiments cells were washed twice with 0.5 ml DPBS. DPBS was completely aspirated from the wells after the second round of washing.

Preparation of spores: Ten milligrams of B. clausii CSI08, B. megaterium MIT411 and B. coagulans CGI314 spores powders were weighted in 15 ml falcon tubes and resuspended in 10 ml of full culture medium without antibiotics. Suspensions were aliquoted and stored at −20° C. until use. Suspensions were used within 2 weeks upon preparation.

Adhesion assay: 500 μl of spores suspensions (1.3×10⁷-9.2×10⁷ CFU/ml) were added to HT-29 and HT-29-MTX cells, mixed by a gentle swirl, and incubated for 2.5 h at 37° C. in the CO₂ incubator. Control wells not containing mammalian cells were prepared and incubated in parallel in the same way (0.5 ml of spores' suspensions).

Upon incubation HT-29 and HT-29-MTX cells were washed 4 times with 0.5 ml PBS. After that 50 μl of Trypsin/EDTA solution and 50 μl of PBS were added to the wells and incubated for 10 min with gentle shaking (˜100 rpm) at 37° C. Fifty microliters of Trypsin/EDTA solution were added to control wells.

Consequently, 450 μl of PBS were added to the wells with spores, contents of the wells were transferred into Eppendorf tubes with scrapping and subjected to three rounds of vigorous shaking 30 sec each. Contents of control wells were transferred into Eppendorf tubes and subjected to one round of shaking.

Serial dilutions (plus dilutions of control wells) were prepared in PBS and plated onto BC agar (B. coagulans CGI314) or PetriFilm™ (B. clausii CSI08, B. megaterium MIT411). Plates were incubated at 37° C. for 48 h prior to counting, PetriFilm were incubated at 37° C. for 24 h prior to counting.

Experiments were performed two or three times with three technical replicates per experiment. The results are expressed as means±SEM.

TABLE 8
Adherence of B. clausii CSI08, B. megaterium MIT411 and
B. coagulans CGI314 spores to the HT-29-MTX cell line

| Percentage of | | | |
|---|---|---|---|
| adherence to HT- | B. clausii | B. megaterium | B. coagulans |
| 29-MTX cell line | CSI08 | MIT411 | CGI314 |
| Mean | 12.10 | 20.68 | 19.68 |
| Standard error of | 0.7710 | 3.005 | 2.197 |
| the mean | | | |

TABLE 9
Adherence of B. clausii CSI08, B. megaterium MIT411
and B. coagulans CGI314 spores to the HT-29 cell line

| Percentage of | | | |
|---|---|---|---|
| adherence to HT- | B. clausii | B. megaterium | B. coagulans |
| 29 cell line | CSI08 | MIT411 | CGI314 |
| Mean | 0.2578 | 1.499 | 0.8033 |
| Standard error of | 0.02035 | 0.2983 | 0.1781 |
| the mean | | | |

### Conclusion:

1. Results set out above demonstrate higher ability of spores to adhere to the mucous-secreting cell line HT-29-MTX compared to non-mucus secreting cells, possibly due to spores' physical properties.
2. B. megaterium MIT411 and B. coagulans CGI314 spores have higher (but overall low) ability to adhere to non-mucus producing cell line HT-29 compared to B. clausii CSI08 spores.

### Example 3

### Evaluation of Bacillus clausii CSI08, Bacillus megaterium MIT411 and a Bacillus Cocktail on Safety, Tolerance and Gastrointestinal Health: A Randomised, Double-Blind, Placebo-Controlled Trial in Healthy Adults

The safety, tolerance and impact of 1×10⁹ CFU Bacillus clausii CSI08, 1×10⁹ CFU Bacillus megaterium MIT411 and a probiotic cocktail containing 0.5×10⁹ CFU of Bacillus subtillis DE111®, 0.5×10⁹ CFU of Bacillus megaterium MIT411, 0.5×10⁹ CFU Bacillus coagulant CGI314, 0.5×10⁹ CFU Bacillus clausii CSI08 (i.e., Bacillus subtilis DE111®, Bacillus megaterium MIT411, Bacillus coagulans CGI314, and Bacillus clausii CSI08 with a total count of 2.0×10⁹ CFU) administered daily were assessed as compared with a maltodextrin containing placebo control. A total of 98 study participants received daily doses for 45 days, followed by a washout period of 2 weeks. A questionnaire to capture the incidence and duration of upper respiratory tract, urinary tract and/or gastrointestinal complaints and a diary to capture stool regularity and consistency was kept daily to record compliance throughout the 45 days. Faecal and blood samples were collected for microbiological and haematological analysis at the start and end of the treatment period. The probiotic cocktail significantly decreased the incidence of loose stools throughout the entire study. The recorded respiratory, urinary and gastrointestinal symptoms, defecation frequency and other stool consistency were not influenced. No clinically relevant changes in blood parameters such as liver and kidney function and no serious adverse events appeared during and after administration. There were no changes in symptoms including sadness, irritability, energy, appetite, tension, stress, sleep, cardiovascular events, aches and pains, and dizziness as determined by a mood questionnaire administered to participants at baseline and at the end of the treatment period. Similarly, the measured inflammatory cytokines, antioxidant levels, cholesterol, triglycerides, free amino acids or minerals remained unaffected. There were no negative changes in alpha or beta diversity of the microbiota with any of the treatment groups. These promising data suggest that these treatments were safe and well tolerated, and further work with larger cohorts are justified to determine the efficacy of these potential probiotics in select demographic groups.

Probiotics are live microorganisms residing in the human gut with low or no pathogenicity and exhibit beneficial effects for the host. Common products containing probiotic bacteria include dietary supplements and foodstuffs such as fermented dairy products, sauerkraut, and salami. Probiotic supplementation has shown positive results for relief of various ailments such as: antibiotic associated diarrhea, constipation, allergies, and diabetes. Probiotics have also exhibited protective properties.

Probiotic supplements can contain one or more different bacterial strains that exert different effects on the human gut. Common probiotic strains are lactic acid producers such as Lactobacillus, Bifidobacterium, and Streptococcus due to their resistance to gastric acids, bile salts, and pancreatic enzymes. Studies have shown that lactic acid bacteria are effective inhibitors of pathogenic, gram-negative, bacterial colonization (e.g. Salmonella typhimurium, Clostridium difficile, and Escherichia coli) in vitro.

Not all probiotic supplements are lactic acid producers however. Bacillus subtilis spores have been used as probiotics, competitive exclusion agents, and prophylactics for human and animal consumption. All four Bacilli strains are gram-positive, spore forming, rod-shaped bacterium. Under nutrient limiting conditions, Bacillus sp. can form resistant dormant endospores to environmental stressors and nutrient deprivation, making these bacteria a viable option for a probiotic supplement.

DE111, CSI08, CGI314, and MIT411 are unique strain of probiotics. Being Bacillus strains of probiotics, they are able to resist the harsh digestive environment and colonise the gut, thus supporting a healthy GI tract. To date, DE111 is sold in both the USA and Canada as a probiotic food ingredient and as a probiotic capsule for adults. The other three Bacillus probiotics CSI08, CGI314, and MIT411 used in this trial are not currently on the market and are claimed herein.

This trial was to determine the safety of 3 new probiotic strains and to assess their efficacy in reducing the incidence and/or duration of gastrointestinal problems and infections as well as respiratory infections in healthy adults.

### Materials and Methods

### Subjects

Healthy adult volunteers, 18-65 years of age, were recruited using flyers, posters and from their physicians from February to July 2021. Inclusion criteria included: willingness to provide informed consent and being in good overall health. Exclusion criteria included: existence of any pre-existing adverse event conditions (e.g. gastric ulcer, Crohn's disease, UC, diabetes, kidney disease, HIV/AIDS, hepatitis, cancer, and organ transplant recipient), taking medications for digestive complaints (constipation, bloating or diarrhoea), antibiotic usage within the past four weeks prior to randomisation, unwillingness to discontinue any probiotic supplement other than that provided by the study, known immunodeficiency or use of immunosuppressive medication, pregnancy, 6 month post-partum or breastfeeding, women of childbearing age planning on pregnancy during the course of the study, participation in another study and use of medication for mood (e.g. antidepressants, anxiolytics, antipsychotics).

This study was approved by the University of Ljubljana, Biotechnical Faculty, Nutritional Research Ethics Committee in Slovenia and conducted according to guidelines established by the Declaration of Helsinki. All participants were informed of the aims, requirements and risks of the study in addition to being notified that they could withdraw from the study at any time. Participants provided their written consent indicating their full knowledge of the study protocol.

### Experimental Design

This study was a double-blinded, placebo-controlled, randomized, parallel trial. The study took place through University Clinical Centre Maribor, Slovenia and was co-ordinated by the CRO Vizera d.o.o., Slovenia. Participants were randomised to either one of three treatment groups or placebo administered daily. Treatment groups were 1×10⁹ CFU/dose of Bacillus clausii CSI08, 1×10⁹ CFU/dose of Bacillus megaterium MIT411, and a probiotic cocktail containing Bacillus subtilis DE111®, Bacillus megaterium MIT411, Bacillus coagulans CGI314, and Bacillus clausii CSI08 with a total count of 2.0×10⁹ CFU/dose administered daily. Placebo was rice maltodextrin.

A randomisation scheme was performed by CRO Vizera d.o.o., Slovenia with the allocation sequence being concealed from study personnel and participants until randomisation day in sealed, opaque envelopes. After assessment of baseline characteristics (age, sex, height, weight by digital scale) and collection of an initial stool sample, an envelope was unscaled and participants were assigned to an intervention. Investigators received individually closed envelopes containing the link between the randomization number and the treatment group for a specific participant. The sealed envelopes could only be opened in case of emergency. The Sponsor was immediately notified if a participant's treatment was unblinded during the course of the study. Information regarding the un-blinding had to be recorded in the data source document and in the Case Report Form (CRF) of the participant. Participants were then instructed to consume one capsule per day at the end of a meal.

Participants visited the study centre 3 times, and performed 2 calls with the designated Investigator: Visit 0 for screening purposes (Screening Visit), 2 times during treatment period with Visit 1 being baseline visit, where randomization and distribution of product were performed, and Visit 2 being the End of Treatment Visit. Additionally, the patients performed a phone call with Investigator after 21 days of product consumption (In between visits call) and after 2 weeks of follow up following Visit 2 (Follow-up call). A graphical flow chart of the study is presented in FIG. 25.

After screening, consent and randomization, participants provided blood and stool samples prior to any treatment. At the end of the 45 day intervention period, study participants provided a second stool sample and again provided blood samples.

FIG. 25 shows a graphical flow chart of the study design.

### Probiotic Administration Protocol

Deerland Probiotics and Enzymes (Kennesaw, Georgia, US) provided investigational products as identical, oblong 300 mg capsules and placebo was indistinguishable by appearance. The study capsules were provided in bottles labelled with a treatment code by a study collaborator who did not have contact with study personnel or participants.

### Study Protocol

Participants completed the questionnaire daily to monitor time of defecation and type of stool samples based on the Bristol stool chart index and if there were any symptoms including: gastrointestinal distress, respiratory distress, urinary tract symptoms, cephalic, car-nose-throat, behavioural, emetic, loss of appetite, fever and epidermal. If any visits to their GP or any medication was prescribed during the trial this was also captured and reported. A mood questionnaire was administered to participants at baseline and at the end of the treatment period to assess their experience over the previous month. This questionnaire consisted of 14 captured symptoms including sadness, irritability, energy, appetite, tension, stress, sleep, cardiovascular events, aches and pains and dizziness on a scale of 1 (no noticeable symptoms) to 3 (severe). Any adverse events were reported to study staff.

### Blood Samples Collection and Preparation

For safety bloods, a 3-mL red cap serum clot activator tube was used (Greiner Bio-One, 454029) for blood collection. For biochemistry blood panel high- and low-density lipoproteins, total cholesterol and triglyceride determination, 3.5 mL SST II Advanced/gel yellow cap vials (Greiner Bio-One, 454029) were used. For antioxidants and cytokine determination, whole blood was collected into 4-mL lithium-heparin containing tubes (Greiner Bio-One, 454029). Plasma samples were prepared by centrifugation at 2000 G for 15 min. The supernatant was aliquoted and stored at −80° C. for later analysis.

### LDL, HDL, Total Cholesterol and Triglyceride Determination

Hematology and Biochemistry assessment were run in University Clinical Centre Maribor, Slovenia. Safety bloods were run with a Sysmex EN-1000, while Biochemical assays for LDL, HDL, total cholesterol and triglyceride were assayed according to manufacturer's instructions and analysed with an Abbott Allinity C.

### Cytokine Quantification

The concentrations of IL-8 and TNF-alpha in serum samples were determined by sandwich ELISAs: Human IL-8 (CXCL8) ELISA Kit (ELH-IL8-1, RayBiotech) and Human TNF alpha ELISA Kit (ELH-TNFa-1, RayBiotech) according to the manufacturer's instruction. Prior to ELISAs serum samples were diluted 1:2 using dilution buffers supplied with the kits.

### Antioxidant Activity Determination

Total antioxidant activity was assessed using the total antioxidant capacity assay kit (Sigma, Ireland) according to manufacturer's instructions and the absorbance was measured at 340 nm.

### Stool Collection

Stools were collected at the baseline visit prior to treatment and again at the final visit on day 45 using Zymokit DNA/RNA Shield™ Fecal Collection Tube (ZymoResearch, California, US). Participants were instructed to place the collection systems containing the samples on ice immediately after defecation and to deliver samples to study personnel on clinic visits. DNA extraction and 16S rRNA sequencing

Total fecal DNA from approximately 200 mg sample was extracted using ZymoBIOMICS DNA Miniprep Kit (Zymo Research, Irvine, CA, USA) in accordance with manufacturer's instructions. Briefly, the stool samples were placed in the ZR BashingBead™ Lysis tubes containing 750 μl ZymoBIOMICS™ Lysis Solution and processed in a BeadBug™ 6 homogenizer (Benchmark Scientific, China): 5×1 min beating at 4350 rpm with 1 min intermittent step between beating cycles. After that, the lysis tubes were centrifuged at 10,000 g for 1 minute. Four hundred microliters of supernatants were transferred to the Zymo-Spin™ III-F Filters in collection tubes and further centrifuged at 8,000 g for 1 minute. The filtrates were mixed with 1,200 μl of ZymoBIOMICS™ DNA Binding Buffer, transferred to Zymo-Spin™ IICR Columns in Collection Tubes and centrifuged at 10,000 g for 1 minute. After three rounds of washing, DNA was eluted in 100 μl of ZymoBIOMICS™ DNase/RNase Free water and further purified using Zymo-Spin™ III-HRC Filters according to the protocol. DNA concentration was determined using Qubit dsDNA BR Assay kit (ThermoFisher Scientific).

### Data Generation

Library preparation was performed following the Illumina guidelines for 16S Metagenomic Sequencing Library Preparation (https://support.illumina.com/documents/documentation/chemistry_documentation/16s/16s-metagenomic-library-prep-guide-15044223-b.pdf). Briefly, 16S degenerated primers are used to amplify the target from each sample. At the same time Illumina adapters and barcodes are included to allow the creation of the library. Sequencing was performed on a Novaseq 6000 machine producing paired-end 250 bp reads. A quality control of the sequencing data was performed with the software QIIME2. On average, 670 thousand read pairs were produced per sample. Taxonomic classification of the ASVs (also referred to as OTUs) was performed using QIIME2/DADA2 and the Silva132 database.

### Statistical Analyses

25 participants per arm was determined to be sufficient to assess the occurrence and nature of possible adverse events including incidence and duration of urinary tract, gastrointestinal, and upper respiratory complaints. Descriptive statistics was used to evaluate these outcomes in this study. Kruskal-Wallis test was used to confirm there was no statistically significant difference in the occurrence of any of these individual symptoms among the four treatment groups at the beginning of the study, or in the incidence and duration of gastrointestinal, upper respiratory or urinary tract complaints over the duration of the study. Furthermore, nonparametric Mann-Whitney U test with Holm's correction was used for pairwise comparison between each of the three probiotic product groups compared to placebo group.

For the gastrointestinal health questionnaire and blood analyses the difference in individual symptoms score change from baseline to the end of the treatment period was compared among the treatment groups using the Analysis of variance (one-way ANOVA test) with post hoc test evaluating pairwise comparison between each of the three treatment groups as compared to placebo group.

For sequencing data, multiple alpha diversity indices were calculated including Observed, Chao1, ACE, Shannon and Simpson index. The alpha diversity was then compared among the experimental groups and against the Placebo in order to detect differences due to the treatments- or within treatments from baseline to the post-treatment timepoint.

With the aim of quantifying compositional dissimilarity between different samples, the Bray-Curtis dissimilarity index was calculated and used for the creation of multiple clustering plots. This method collapses information from multiple dimensions for ease of visualisation and interpretation. A paired Wilcoxon test was used to compare the distribution of the groups.

Differential abundance analyses were carried out to detect significant differences in genera abundance across the different treatments and time points. Day 1 samples from all treatments were compared against the Day 1 Placebo group to determine if there were any resting difference at baseline. For each group, the pairwise comparison Day 45 vs Day 1 was performed. Bifactorial analyses was also performed using the Placebo group as reference to detect if there is a significant difference in the response of the treatments at Day 45 with respect to Day 1 compared to the response of the Placebo group at Day 45 with respect to Day 1.

### Results

### Participants

Ninety eight participants completed the 45 day intervention (FIG. 25). After screening, one participant declined to participate, and another was withdrawn as they became pregnant. A total of 12 adverse events were reported in the study. These included gastroesophageal reflux (3 AEs), rash (2 AEs), and vertigo (2 AEs). One case of rash was reported as a fungal rash (Tinea corporis) and one case of vertigo was attributed to the use of approved co-medication. All other reported AEs occurred only once, namely: vaginal inflammation, stool parasite (possibly related to a trip overseas), right wrist spin, metallic taste, lower back pain, inflammation of sebaceous gland, granuloma, dark brown coloured stool, and acne.

Causality assessment revealed no relation between the reported AEs and the study products.

No serious adverse events were reported throughout the study.

### Participant Demographics:

TABLE 10
Participant demographics for the study

| | | | | | | | Probiotic | |
|---|---|---|---|---|---|---|---|---|
| | Placebo | | B. clausii | | B. megaterium | | cocktail | |
| | N = 24 | | N = 24 | | N = 25 | | N = 25 | |
| Male (%) | 7 | (29.2) | 9 | (37.5) | 7 | (28) | 7 | (28) |
| Female (%) | 17 | (70.8) | 15 | (62.5) | 18 | (72) | 18 | (72) |
| Mean Age (SD) | 40.5 | (12.3) | 37.3 | (12.7) | 40.6 | (9.6) | 35 | (11.6) |
| Age (min-max) | 22-63 | | 19-65 | | 23-58 | | 18-60 | |
| Mean Height (SD) | 172 | (0.1) | 172 | (0.1) | 172 | (0.08) | 171 | (0.09) |
| Height (min-max) | 152-189 | | 158-192 | | 160-186 | | 155-190 | |
| Mean Weight (SD) | 73.9 | (16.2) | 70.7 | (16.1) | 78.1 | (15.8) | 70.2 | (14.7) |

| | | | | |
|---|---|---|---|---|
| Weight (min-max) | 52-112 | 48-106 | 45-120 | 50-101.5 |

### Gastrointestinal Health Status at Screening Visit:

TABLE 11
Gastrointestinal health at baseline (N = 98). (How often
have you had the following problems during last month?)

| | | | | | | | Probiotic | |
|---|---|---|---|---|---|---|---|---|
| | Placebo | | B. clausii | | B. megaterium | | cocktail | |
| | (N* = 24) | | (N* = 24) | | (N* = 25) | | (N* = 25) | |
| Diarrhea (p = 0.499) | 24 | | 24 | | 25 | | 25 | |
| No problems or <1/month (%) | 22 | (91.7) | 24 | (100.0) | 23 | (92.0) | 24 | (96.0) |
| Monthly basis (%) | 2 | (8.3) | 0 | (0.0) | 2 | (8.0) | 1 | (4.0) |
| Weekly basis (%) | 0 | (0.0) | 0 | (0.0) | 0 | (0.0) | 0 | (0.0) |
| Daily basis (%) | 0 | (0.0) | 0 | (0.0) | 0 | (0.0) | 0 | (0.0) |
| Loose stool (p = 0.771) | 24 | | 24 | | 25 | | 25 | |
| No problems or <1/month (%) | 12 | (50.0) | 16 | (66.7) | 16 | (64.0) | 15 | (60.0) |
| Monthly basis (%) | 10 | (41.7) | 6 | (25.0) | 7 | (28.0) | 8 | (32.0) |
| Weekly basis (%) | 1 | (4.2) | 1 | (4.2) | 1 | (4.0) | 0 | (0.0) |
| Daily basis (%) | 1 | (4.2) | 1 | (4.2) | 1 | (4.0) | 2 | (8.0) |
| Constipation (p = 0.625) | 24 | | 24 | | 25 | | 25 | |
| No problems or <1/month (%) | 16 | (66.7) | 18 | (75.0) | 20 | (80.0) | 20 | (80.0) |
| Monthly basis (%) | 6 | (25.0) | 4 | (16.7) | 4 | (16.0) | 3 | (12.0) |
| Weekly basis (%) | 0 | (0.0) | 1 | (4.2) | 1 | (4.0) | 2 | (8.0) |
| Daily basis (%) | 2 | (8.3) | 1 | (4.2) | 0 | (0.0) | 0 | (0.0) |
| Bowel sounds/stomach | 24 | | 24 | | 25 | | 25 | |
| rumble (p = 0.745) | | | | | | | | |
| No problems or <1/month (%) | 13 | (54.2) | 16 | (66.7) | 17 | (68.0) | 18 | (72.0) |
| Monthly basis (%) | 8 | (33.3) | 5 | (20.8) | 5 | (20.0) | 3 | (12.0) |
| Weekly basis (%) | 3 | (12.5) | 2 | (8.3) | 3 | (12.0) | 4 | (16.0) |
| Daily basis (%) | 0 | (0.0) | 1 | (4.2) | 0 | (0.0) | 0 | (0.0) |
| Cramping/stomach pain (p = 0.880) | 24 | | 24 | | 25 | | 25 | |
| No problems or <1/month (%) | 18 | (75.0) | 17 | (70.8) | 21 | (84.0) | 18 | (72.0) |
| Monthly basis (%) | 4 | (16.7) | 6 | (25.0) | 2 | (8.0) | 6 | (24.0) |
| Weekly basis (%) | 2 | (8.3) | 0 | (0.0) | 2 | (8.0) | 1 | (4.0) |
| Daily basis (%) | 0 | (0.0) | 1 | (4.2) | 0 | (0.0) | 0 | (0.0) |
| Flatulence (p = 0.153) | 24 | | 24 | | 25 | | 25 | |
| No problems or <1/month (%) | 12 | (50.0) | 9 | (37.5) | 7 | (28.0) | 16 | (64.0) |
| Monthly basis (%) | 6 | (25.0) | 9 | (37.5) | 10 | (40.0) | 5 | (20.0) |
| Weekly basis (%) | 4 | (16.7) | 5 | (20.8) | 3 | (12.0) | 3 | (12.0) |
| Daily basis (%) | 2 | (8.3) | 1 | (4.2) | 5 | (20.0) | 1 | (4.0) |
| Bloating (p = 0.136) | 24 | | 24 | | 25 | | 25 | |
| No problems or <1/month (%) | 12 | (50.0) | 14 | (58.3) | 9 | (36.0) | 15 | (60.0) |
| Monthly basis (%) | 8 | (33.3) | 7 | (29.2) | 9 | (36.0) | 8 | (32.0) |
| Weekly basis (%) | 3 | (12.5) | 3 | (12.5) | 4 | (16.0) | 2 | (8.0) |
| Daily basis (%) | 1 | (4.2) | 0 | (0.0) | 3 | (12.0) | 0 | (0.0) |
| Blood in stool (p = 0.393) | 24 | | 24 | | 25 | | 25 | |
| No (%) | 23 | (95.8) | 23 | (95.8) | 24 | (96.0) | 22 | (88.0) |
| Rarely (%) | 0 | (0.0) | 1 | (4.0) | 2 | (8.0) | 0 | (0.0) |
| Occasionally (%) | 1 | (4.2) | 0 | (0.0) | 0 | (0.0) | 1 | (4.2) |
| Often (%) | 0 | (0.0) | 0 | (0.0) | 1 | (4.0) | 0 | (0.0) |

N* = number of participants included in the ITT population,
*p-value for Kruskal-Wallis test

There were no significant differences between the groups for any of individual readouts.

### Stool Consistency and Regularity

Mean bowel movement frequency (regularity) ranged from 0.33 to 2.16 stools/day in the study participants. A variety of period and intervention group comparisons were concluded not equivalent. Bowel movement frequencies were not significantly different when comparing means to placebo treatment group or washout period (Table 12).

TABLE 12
Treatment had no effect on stool regularity over the
duration of the 45 day trial as compared with placebo

| | | | |
|---|---|---|---|
| | | | Probiotic |
| Placebo | B. clausii | B. megaterium | cocktail |
| (N = 24) | (N = 24) | (N = 25) | (N = 25) |

Stools per day (p = 0.834)*

| | | | | |
|---|---|---|---|---|
| mean (SD): | 1.21 (0.41): | 1.2 (0.39): | 1.1 (0.3): | 1.19 (0.45): |
| min-max | 0.36-2.04 | 0.52-2.13 | 0.51-2.04 | 0.33-2.16 |

*p-value for ANOVA test

Stool consistency is reported as the proportion of participants with loose stool and the proportion of participants with hard stool in the total treatment period. Baseline questionnaire reported no differences in the incidence of loose stool or hard stools/constipation in the study groups as compared to control (Table 2). Participants were asked to report over the last month how often they had loose stools or hard stools/constipation. The scale was as follows 0=never, 1=monthly, 2=weekly, 3=daily.

FIG. 26 shows the probiotic cocktail significantly decreased the incidence of loose stool over the course of the study as compared to placebo control.

Over the course of the first 6 weeks of the study, the probiotic cocktail significantly decreased the incidence of loose stools as an overall effect when compared with control (FIG. 26), as determined by repeated measures one way ANOVA (treatment: F (2.615, 13.08)=20.07, P<0.0001; time (F_{(5, 15)}=2.803, p=0.055). Of the study participants, 16 of the 25 in the probiotic group reported no loose stools at all over the course of the study, while there were only 8 in the placebo group, 10 in the B. clausii group, and 10 in the B. megaterium group.

FIG. 27 shows no effect of any treatments on percentage of hard stools as compared to placebo control.

There was no significant effect of any of the treatment groups on the percentage of hard stools over the course of the study (FIG. 27) (F (1.829, 9.146)=2.831, P=0.113; time (F_{(5, 15)}=1.121, p=0.391).

### Incidence and Duration of Gastrointestinal Tract Symptoms

TABLE 13
Number of days with symptoms of gastrointestinal distress
reported in Participant diary over the course of the study

| | | | | | B. | | Probiotic | |
|---|---|---|---|---|---|---|---|---|
| | Placebo | | B. clausii | | megaterium | | cocktail | |
| | (N* = 24) | | (N* = 24) | | (N* = 25) | | (N* = 25) | |
| Loss of appetite | 24 | | 24 | | 25 | | 25 | |
| (p* = 0.233) | | | | | | | | |
| mean (SD): min-max | 0.1 (0.3): | 0-1 | 0.2 (1.0): | 0-5 | 0.0 (NC): | 0-0 | 0.0 (NC): | 0-0 |
| p-value vs placebo | / | | 0.589 | | 0.434 | | 0.434 | |
| Diarrhea (p* = 0.888) | 24 | | 24 | | 25 | | 25 | |
| mean (SD): min-max | 0.1 (0.3): | 0-1 | 0.4 (1.3): | 0-6 | 0.1 (0.3): | 0-1 | 0.2 (0.6): | 0-3 |
| p-value vs placebo | / | | 1.000 | | 1.000 | | 1.000 | |
| Constipation (p* = 0.722) | 24 | | 24 | | 25 | | 25 | |
| mean (SD): min-max | 0.5 (2.4): | 0-12 | 0.3 (1.4): | 0-7 | 0.3 (1.4): | 0-7 | 0.6 (2.2): | 0-8 |
| p-value vs placebo | / | | 0.976 | | 1.000 | | 1.000 | |
| Vomiting (p* = 0.713) | 24 | | 24 | | 25 | | 25 | |
| mean (SD): min-max | 0.1 (0.3): | 0-1 | 0.0 (NC): | 0-0 | 0.0 (0.2): | 0-1 | 0.0 (0.2): | 0-1 |
| p-value vs placebo | / | | 0.611 | | 1.000 | | 1.000 | |
| Gases (p* = 0.468) | 24 | | 24 | | 25 | | 25 | |
| mean (SD): min-max | 0.6 (1.5): | 0-6 | 0.4 (1.1): | 0-5 | 0.9 (1.7): | 0-6 | 0.2 (0.6): | 0-2 |
| p-value vs placebo | / | | 0.639 | | 1.000 | | 1.000 | |
| Bowel sounds (p* = 0.809) | 24 | | 24 | | 25 | | 25 | |
| mean (SD): min-max | 0.2 (0.6): | 0-2 | 0.4 (1.6): | 0-8 | 0.1 (0.3): | 0-1 | 0.2 (0.7): | 0-3 |
| p-value vs placebo | / | | 1.000 | | 1.000 | | 1.000 | |
| Cramping/stomach pain | 24 | | 24 | | 25 | | 25 | |
| (p* = 0.391) | | | | | | | | |
| mean (SD): min-max | 0.5 (1.2): | 0-5 | 0.5 (1.3): | 0-5 | 0.3 (0.6): | 0-2 | 0.1 (0.3): | 0-1 |
| p-value vs placebo | / | | 0.987 | | 1.000 | | 1.000 | |
| Bloating (p* = 0.543) | 24 | | 24 | | 25 | | 25 | |
| mean (SD): min-max | 1.5 (5.1): | 0-25 | 0.3 (1.1): | 0-5 | 0.3 (0.7): | 0-2 | 0.7 (2.1): | 0-8 |

| | | | | |
|---|---|---|---|---|
| p-value vs placebo | / | 0.558 | 1.000 | 0.963 |

N* = number of participants included in the ITT population, NC = not calculable, p* = p-value for Kruskal-Wallis test, p = p- value for Mann-Whitney U test.

Kruskal-Wallis test did not show significant differences in the number of days with gastrointestinal infection symptoms among treatment groups. Compared to placebo, none of the study products containing probiotics showed a statistically significant difference in the number of days with gastrointestinal distress symptoms.

### Incidence and Duration of Urinary Tract Symptoms

TABLE 14
Number of days with symptoms of urinary tract complaints reported in Participant diary

| | | | | | | | Probiotic | |
|---|---|---|---|---|---|---|---|---|
| | Placebo | | B. clausii | | B. megaterium | | cocktail | |
| | (N* = 24) | | (N* = 24) | | (N* = 25) | | (N* = 25) | |
| Pain burning when | 24 | | 24 | | 25 | | 25 | |
| urinating (p* = 0.257) | | | | | | | | |
| mean (SD): min-max | 0.2 (0.8): | 0-4 | 0.0 (NC): | 0-0 | 0.0 (NC): | 0-0 | 0.0 (NC): | 0-0 |
| p-value vs placebo | / | | 0.635 | | 1.000 | | 1.000 | |
| Higher frequency | 24 | | 24 | | 25 | | 25 | |
| urinating (p* = 0.744) | | | | | | | | |
| mean (SD): min-max | 0.0 (NC): | 0-0 | 0.0 (NC): | 0-0 | 0.2 (0.8): | 0-4 | 0.1 (0.4): | 0-2 |
| p-value vs placebo | / | | 1.000 | | 0.982 | | 0.982 | |
| Cramping pressure lower | 24 | | 24 | | 25 | | 25 | |
| abdomen or back (p* = 0.254) | | | | | | | | |
| mean (SD): min-max | 0.0 (NC): | 0-0 | 0.0 (NC): | 0-0 | 0.2 (0.8): | 0-3 | 0.1 (0.4): | 0-2 |

| | | | | |
|---|---|---|---|---|
| p-value vs placebo | / | 1.000 | 0.646 | 0.982 |

N* = number of participants included in the ITT population, NC = not calculable, p* = p-value for Kruskal-Wallis test, p = p- value for Mann-Whitney U test.

Kruskal-Wallis test did not show any significant differences in the number of days with urinary tract infection symptoms among treatment groups. Compared to placebo, none of the study products containing probiotics showed a statistically significant difference in the number of days with urinary infection symptoms.

### Incidence and Duration of Upper Respiratory Tract Infection

TABLE 15
Number of days with symptoms of respiratory tract complaints reported in Participant diary

| | Placebo | | B. clausii | | B. megaterium | | Probiotic | |
|---|---|---|---|---|---|---|---|---|
| | (N* = 24) | | (N* = 24) | | (N* = 25) | | cocktail (N* = 25) | |
| Fever (p* = 0.935) | 24 | | 24 | | 25 | | 25 | |
| mean (SD): min-max | 0.2 (0.8): | 0-4 | 0.3 (1.3): | 0-6 | 0.2 (0.7): | 0-3 | 0.3 (1.4): | 0-7 |
| p-value vs placebo | / | | 1.000 | | 0.966 | | 1.000 | |
| Headache (p* = 0.898) | 24 | | 24 | | 25 | | 25 | |
| mean (SD): min-max | 1.0 (2.0): | 0-7 | 0.9 (1.7): | 0-6 | 0.6 (1.0): | 0-4 | 0.4 (1.0): | 0-4 |
| p-value vs placebo | / | | 1.000 | | 0.860 | | 1.000 | |
| Stuffed nose (p* = 0.909) | 24 | | 24 | | 25 | | 25 | |
| mean (SD): min-max | 0.3 (1.0): | 0-4 | 0.3 (1.1): | 0-5 | 0.6 (2.2): | 0-10 | 0.4 (2.0): | 0-10 |
| p-value vs placebo | / | | 1.000 | | 0.689 | | 1.000 | |
| Runny nose (p* = 0.530) | 24 | | 24 | | 25 | | 25 | |
| mean (SD): min-max | 0.5 (1.6): | 0-6 | 0.4 (1.2): | 0-5 | 0.8 (2.1): | 0-10 | 0.4 (2.0): | 0-10 |
| p-value vs placebo | / | | 0.957 | | 1.000 | | 1.000 | |
| Sore throat (p* = 0.142) | 24 | | 24 | | 25 | | 25 | |
| mean (SD): min-max | 0.5 (1.1): | 0-4 | 0.2 (0.8): | 0-4 | 0.4 (0.9): | 0-3 | 0.6 (2.1): | 0-10 |
| p-value vs placebo | / | | 0.539 | | 0.870 | | 1.000 | |
| Dry cough (p* = 0.868) | 24 | | 24 | | 25 | | 25 | |
| mean (SD): min-max | 0.6 (1.9): | 0-8 | 0.1 (0.3): | 0-1 | 0.4 (2.0): | 0-10 | 0.1 (0.6): | 0-3 |
| p-value vs placebo | / | | 1.000 | | 0.607 | | 1.000 | |
| Productive cough (p* = 0.395) | 24 | | 24 | | 25 | | 25 | |
| mean (SD): min-max | 0.1 (0.4): | 0-2 | 0.0 (NC): | 0-0 | 0.4 (2.0): | 0-10 | 0.4 (1.6): | 0-8 |
| p-value vs placebo | / | | 0.459 | | 1.000 | | 0.983 | |
| Sore ear (p* = 0.417) | 24 | | 24 | | 25 | | 25 | |
| Mean (SD): min-max | 0.0 (NC): | 0-0 | 0.0 (NC): | 0-0 | 0.0 (NC): | 0-0 | 0.0 (0.2): | 0-1 |

| | | | | |
|---|---|---|---|---|
| p-value vs placebo | / | 1.000 | 1.000 | 1.000 |

N* = number of participants included in the ITT population, NC = not calculable, p* = p-value for Kruskal-Wallis test, p = p- value for Mann-Whitney U test.

Kruskal-Wallis test did not show any significant differences in the number of days with respiratory tract infection symptoms among treatment groups. Compared to placebo, none of the study products containing probiotics showed a statistically significant difference in the number of days with symptoms.

### Daily Questionnaire Analysis

Table 16 summarizes the answers to the Mood questionnaire at baseline and at the end of the study for the 3 treatment groups and the placebo. Mean changes with 95% confidence interval are shown. Results of the ANOVA omnibus test (p*-value) and one-sample T test (p-value) are also presented. Test of normality for the change in scores of the Gut-brain axis show that the data do not follow normal distribution, which could affect the results with borderline significance (p-values between 0.05 and 0.10). This affects two items: Loss of energy and Changes in appetite. An alternative nonparametric Kruskal Wallis test was applied to these items; p-values of 0.111 (Loss of energy) and 0.123 (Changes in appetite) were observed. In general, mean values of the scores were less intense (participants were less bothered by these symptoms) at the end of the treatment period including the placebo group. Consequently, One-sample T-test results show that in one third of tests (of 70 performed) a statistically significant change Gut-brain axis questionnaire score was observed. However, this can be observed for all treatment groups including the placebo group. Consequently, the results of the ANOVA test show, that no significant differences in Gut-brain axis score change among the treatment groups were detected, however a borderline significance for the items Loss of energy and Changes in appetite was observed. The participants in the Bacillus megaterium group experienced the largest change for these two items. Nevertheless, no statistically significant difference for pairwise comparison of probiotic groups with placebo was observed (Table 16 (below)).

TABLE 16
Gut-brain axis questionnaire answers at baseline (N = 98) and at the end of the study
(Post). (How much did the following emotion or feeling bother you in the last 30 days, including
today?)

| Placebo | | B. clausii | | B. megaterium | | Probiotic cocktail | |
|---|---|---|---|---|---|---|---|
| (N* = 24) | | (N* = 24) | | (N* = 25) | | (N* = 25) | |
| Baseline | Post | Baseline | Post | Baseline | Post | Baseline | Post |

Sadness (p* = 0.264)

| | | | | | | | | |
|---|---|---|---|---|---|---|---|---|
| sum of scores | 12 | 11 | 22 | 13 | 16 | 12 | 16 | 4 |
| No. of participants | 9 | 9 | 14 | 11 | 11 | 8 | 11 | 4 |
| affected | | | | | | | | |

Irritation (p* = 0.341)

| | | | | | | | | |
|---|---|---|---|---|---|---|---|---|
| sum of scores | 21 | 14 | 28 | 20 | 25 | 16 | 28 | 12 |
| No. of participants | 16 | 10 | 19 | 14 | 20 | 12 | 21 | 10 |
| affected | | | | | | | | |

Loss of energy (p* = 0.076)

| | | | | | | | | |
|---|---|---|---|---|---|---|---|---|
| sum of scores | 29 | 18 | 28 | 22 | 37 | 14 | 30 | 18 |
| No. of participants | 19 | 14 | 17 | 15 | 21 | 10 | 20 | 11 |
| affected | | | | | | | | |

Changes in appetite (p* = 0.065)

| | | | | | | | | |
|---|---|---|---|---|---|---|---|---|
| sum of scores | 8 | 7 | 12 | 12 | 19 | 6 | 7 | 5 |
| No. of participants | 6 | 6 | 9 | 10 | 13 | 4 | 5 | 4 |
| affected | | | | | | | | |

Hard to breathe/choking (p* = 0.129)

| | | | | | | | | |
|---|---|---|---|---|---|---|---|---|
| sum of scores | 4 | 4 | 5 | 3 | 7 | 0 | 5 | 1 |
| No. of participants | 4 | 3 | 3 | 3 | 5 | 0 | 5 | 1 |
| affected | | | | | | | | |

Heart pounding/racing (p* = 0.744)

| | | | | | | | | |
|---|---|---|---|---|---|---|---|---|
| sum of scores | 7 | 5 | 6 | 4 | 8 | 2 | 3 | 3 |
| No. of participants | 6 | 3 | 4 | 3 | 5 | 2 | 3 | 3 |
| affected | | | | | | | | |

Sleeping problems (p* = 0.915)

| | | | | | | | | |
|---|---|---|---|---|---|---|---|---|
| sum of scores | 18 | 14 | 30 | 16 | 21 | 12 | 19 | 11 |
| No. of participants | 12 | 10 | 15 | 11 | 15 | 10 | 14 | 8 |
| affected | | | | | | | | |

Concentration problems (p* = 0.915)

| | | | | | | | | |
|---|---|---|---|---|---|---|---|---|
| sum of scores | 18 | 8 | 28 | 15 | 20 | 8 | 16 | 5 |
| No. of participants | 14 | 8 | 18 | 13 | 15 | 6 | 9 | 3 |
| affected | | | | | | | | |

Nervousness/stress (p* = 0.937)

| | | | | | | | | |
|---|---|---|---|---|---|---|---|---|
| sum of scores | 27 | 17 | 27 | 18 | 24 | 13 | 32 | 18 |
| No. of participants | 18 | 13 | 17 | 13 | 17 | 11 | 20 | 15 |
| affected | | | | | | | | |

Angriness/tension (p* = 0.478)

| | | | | | | | | |
|---|---|---|---|---|---|---|---|---|
| sum of scores | 17 | 11 | 19 | 13 | 19 | 9 | 23 | 18 |
| No. of participants | 13 | 9 | 13 | 9 | 16 | 8 | 18 | 8 |
| affected | | | | | | | | |

Headaches (p* = 0.511)

| | | | | | | | | |
|---|---|---|---|---|---|---|---|---|
| sum of scores | 14 | 8 | 11 | 11 | 20 | 12 | 12 | 5 |
| No. of participants | 10 | 6 | 8 | 8 | 13 | 10 | 10 | 4 |
| affected | | | | | | | | |

Muscle aches/pains (p* = 0.391)

| | | | | | | | | |
|---|---|---|---|---|---|---|---|---|
| sum of scores | 13 | 6 | 9 | 8 | 12 | 5 | 11 | 7 |
| No. of participants | 11 | 5 | 8 | 7 | 8 | 4 | 7 | 4 |
| affected | | | | | | | | |

Stiffness (p* = 0.376)

| | | | | | | | | |
|---|---|---|---|---|---|---|---|---|
| sum of scores | 8 | 2 | 9 | 5 | 12 | 2 | 6 | 4 |
| No. of participants | 6 | 2 | 7 | 4 | 9 | 2 | 5 | 3 |
| affected | | | | | | | | |

Dizziness (p* = 0.732)

| | | | | | | | | |
|---|---|---|---|---|---|---|---|---|
| sum of scores | 5 | 5 | 4 | 2 | 5 | 1 | 1 | 1 |
| No. of participants | 4 | 3 | 3 | 2 | 4 | 1 | 1 | 1 |
| affected | | | | | | | | |

N* = number of participants included in the ITT population; scores: 0-not bothered, 1-mildly bothered, 2-somewhat bothered, and 3-very bothered.
*p-value for ANOVA (omnibus test).

### Cholesterol and Triglyceride Levels

Blood samples were gathered at the start of the study prior to any treatment and again at the end of the 45 day treatment period. There was no significant effect of treatment within groups, nor was there any significant effect of treatment as compared with baseline for high density lipoproteins, low density lipoproteins, total cholesterol and triglyceride concentrations (Table 17.

TABLE 17
Cholesterol and triglyceride levels at baseline,
and at the end of the study (N = 98)

| | | | |
|---|---|---|---|
| | Bacillus | Bacillus | Probiotic |
| Placebo | clausii | megaterium | cocktail |
| (N* = 24) | (N* = 24) | (N* = 25) | (N* = 25) |

HDL [mmol/L] (p = 0.749)

| | | | | |
|---|---|---|---|---|
| Baseline: mean (SD) | 1.4 (0.2): | 1.6 (0.3): | 1.5 (0.4): | 1.6 (0.4): |
| Post: mean (SD) | 1.4 (0.2) | 1.5 (0.3) | 1.4 (0.3) | 1.6 (0.4) |

LDL [mmol/L] (p = 0.894)

| | | | | |
|---|---|---|---|---|
| Baseline: mean (SD) | 3.3 (0.9): | 3.5 (0.8): | 3.7 (1.0): | 3.4 (0.7): |
| Post: mean (SD) | 3.3 (0.9) | 3.4 (0.8) | 3.7 (1.0) | 3.3 (0.7) |

TC [mmol/L] (p = 0.976)

| | | | | |
|---|---|---|---|---|
| Baseline: mean (SD) | 5.3 (1.2): | 5.6 (1.0): | 5.9 (1.5): | 5.5 (0.9): |
| Post: mean (SD) | 5.0 (1.1) | 5.4 (1.0) | 5.6 (1.2) | 5.3 (0.9) |

TG [mmol/L] (p = 0.548)

| | | | | |
|---|---|---|---|---|
| Baseline: mean (SD) | 1.0 (0.6): | 1.0 (0.4): | 1.8 (3.6): | 1.2 (1.2): |
| Post: mean (SD) | 1.2 (0.7) | 1.2 (0.8) | 1.7 (2.6) | 1.3 (1.2) |

N* = number of participants included in the ITT population, HDL = high-density lipoprotein, LDL = low-density lipoprotein, TC = total cholesterol, TG = triglycerides.
*p-value for ANOVA (omnibus test)

### Blood Cytokine Levels

TABLE 18
IL-8 and TNFα levels at baseline, and at the end of the study (N = 98)

| | | | |
|---|---|---|---|
| | Bacillus | Bacillus | Probiotic |
| Placebo | clausii | megaterium | cocktail |
| (N* = 24) | (N* = 24) | (N* = 25) | (N* = 25) |

IL-8 [pg/mL] (p = 0.887)

| | | | | |
|---|---|---|---|---|
| Baseline: mean (SD) | 99.65 (60.87): | 105.4 (59.02): | 100 (58.47): | 94.72 (71.38): |
| Post: mean (SD) | 94.17 (70.96) | 127 (89.38) | 120.3 (82.96) | 146.5 (107.6) |

TNFα [pg/mL] (p = 0.662)

| | | | | |
|---|---|---|---|---|
| Baseline: mean (SD) | 445.2 (322.6): | 490.7 (244.9): | 358.7 (180.4): | 549.3 (492.9): |
| Post: mean (SD) | 452.7 (345.3) | 525.9 (416.8) | 473.2 (331.4) | 573.1 (423) |

N* = number of participants included in the ITT population,
*p-value for ANOVA (omnibus test)

Blood samples were gathered at the start of the study prior to any treatment and again at the end of the 45 day treatment period. There was no significant effect of treatment within groups, nor was there any significant effect of treatment as compared with baseline for IL-8 or TNFα (Table 18).

### Blood Antioxidant Levels

TABLE 19
Antioxidant levels at baseline, and at the end of the study (N = 98)

| | | | |
|---|---|---|---|
| | Bacillus | Bacillus | Probiotic |
| Placebo | clausii | megaterium | cocktail |
| (N* = 24) | (N* = 24) | (N* = 25) | (N* = 25) |

Conc. Of Trolox Equivalent [nmole/mL] (p = 0.831)

| | | | | |
|---|---|---|---|---|
| Baseline: mean (SD) | 3898 (316.5): | 3941 (319.8): | 3855 (366.3): | 3943 (290): |
| Post: mean (SD) | 3919 (483.7) | 4007 (364.3) | 3983 (420.9) | 3916 (361.5) |

N* = number of participants included in the ITT population,
*p-value for ANOVA (omnibus test)

Blood samples were gathered at the start of the study prior to any treatment and again at the end of the 45 day treatment period. There was no significant effect of treatment within groups, nor was there any significant effect of treatment as compared with baseline for antioxidant levels (Table 10).

### Metabolite Levels

TABLE 20
Amino acid levels at baseline, and at the end of the study (N = 98)

| | | | |
|---|---|---|---|
| | Bacillus | Bacillus | Probiotic |
| Placebo | clausii | megaterium | cocktail |
| (N* = 24) | (N* = 24) | (N* = 25) | (N* = 25) |

Aspartate [mg/L] (*p = 0.3752)

| | | | | |
|---|---|---|---|---|
| Baseline: mean (SD) | 1.947 (0.6): | 1.427 (0.3): | 1.382 (0.3): | 2.183 (0.8): |
| Post: mean (SD) | 2.244 (1.1) | 1.526 (0.6) | 1.309 (0.5) | 2.569 (1.0) |

Serine [mg/L] (*p = 0.6652)

| | | | | |
|---|---|---|---|---|
| Baseline: mean (SD) | 16.891 (6.1): | 11.094 (2.7): | 15.203 (1.9): | 14.255 (4.1): |
| Post: mean (SD) | 16.406 (4.3) | 11.625 (3.5) | 15.718 (5.5) | 16.388 (4.5) |

Histidine [mg/L] (*p = 0.8230)

| | | | | |
|---|---|---|---|---|
| Baseline: mean (SD) | 57.285 (14.6): | 48.185 (8.7): | 64.944 (12.2): | 52.170 (11.1): |
| Post: mean (SD) | 59.280 (11.9) | 51.156 (11.2) | 71.494 (20.6) | 59.894 (12.7) |

Arginine [mg/L] (*p = 0.7104)

| | | | | |
|---|---|---|---|---|
| Baseline: mean (SD) | 28.951 (13.2): | 21.006 (7.3): | 25.925 (7.9): | 21.759 (8.8): |
| Post: mean (SD) | 26.675 (11.3) | 22.846 (8.8) | 24.431 (11.6) | 24.413 (11.1) |

Glutamine [mg/L] (*p = 0.8118)

| | | | | |
|---|---|---|---|---|
| Baseline: mean (SD) | 18.254 (7.2): | 10.970 (2.9): | 16.626 (6.6): | 15.973 (6.1): |
| Post: mean (SD) | 17.266 (6.0) | 9.747 (3.3) | 14.395 (6.7) | 16.925 (6.2) |

Glycine [mg/L] (*p = 0.8067)

| | | | | |
|---|---|---|---|---|
| Baseline: mean (SD) | 17.272 (5.0): | 12.164 (4.5): | 20.620 (4.9): | 14.508 (4.2): |
| Post: mean (SD) | 16.450 (4.1) | 11.660 (3.2) | 21.257 (8.0) | 15.903 (3.5) |

Threonine [mg/L] (*p = 0.8875)

| | | | | |
|---|---|---|---|---|
| Baseline: mean (SD) | 12.658 (4.5): | 9.641 (2.1): | 12.005 (2.6): | 11.847 (3.4): |
| Post: mean (SD) | 12.939 (4.1) | 9.574 (2.3) | 12.656 (5.2) | 13.365 (3.7) |

Alanine [mg/L] (*p = 0.5359)

| | | | | |
|---|---|---|---|---|
| Baseline: mean (SD) | 27.476 (7.9): | 19.316 (4.6): | 25.346 (6.1): | 23.329 (6.1): |
| Post: mean (SD) | 25.796 (7.3) | 20.151 (6.4) | 25.924 (9.5) | 26.976 (6.7) |

Proline [mg/L] (*p = 0.5683)

| | | | | |
|---|---|---|---|---|
| Baseline: mean (SD) | 19.101 (6.3): | 13.542 (3.8): | 19.477 (3.0): | 16.487 (4.7): |
| Post: mean (SD) | 19.581 (6.7) | 14.221 (3.6) | 20.163 (6.6) | 20.570 (5.8) |

Tyrosine [mg/L] (*p = 0.4548)

| | | | | |
|---|---|---|---|---|
| Baseline: mean (SD) | 10.617 (4.0): | 7.542 (2.2): | 11.594 (2.1): | 9.326 (3.5): |
| Post: mean (SD) | 10.337 (2.7) | 7.786 (2.0) | 11.759 (3.4) | 11.112 (3.0) |

Valine [mg/L] (*p = 0.8116)

| | | | | |
|---|---|---|---|---|
| Baseline: mean (SD) | 16.720 (4.2): | 12.976 (2.9): | 19.415 (3.1): | 16.746 (4.4): |
| Post: mean (SD) | 17.916 (5.0) | 13.512 (4.3) | 20.359 (4.7) | 19.411 (3.9) |

Methionine [mg/L] (*p = 0.5286)

| | | | | |
|---|---|---|---|---|
| Baseline: mean (SD) | 2.850 (1.0): | 2.117 (0.5): | 2.710 (0.4): | 2.546 (0.8): |
| Post: mean (SD) | 2.926 (0.9) | 2.280 (0.7) | 2.803 (0.9) | 3.037 (0.7) |

Lysine [mg/L] (*p = 0.3400)

| | | | | |
|---|---|---|---|---|
| Baseline: mean (SD) | 22.722 (6.5): | 17.765 (4.1): | 20.404 (4.5): | 22.181 (5.3): |
| Post: mean (SD) | 24.508 (6.5) | 18.470 (6.8) | 20.734 (6.6) | 27.171 (7.1) |

Isoleucine [mg/L] (*p = 0.9065)

| | | | | |
|---|---|---|---|---|
| Baseline: mean (SD) | 6.354 (2.0): | 4.684 (1.1): | 6.754 (1.1): | 6.236 (1.6): |
| Post: mean (SD) | 6.955 (2.2) | 5.149 (1.9) | 7.321 (1.8) | 7.412 (1.7) |

Leucine [mg/L] (*p = 0.6745)

| | | | | |
|---|---|---|---|---|
| Baseline: mean (SD) | 13.418 (5.2): | 9.122 (2.3): | 13.446 (2.0): | 12.612 (3.9): |
| Post: mean (SD) | 13.865 (4.7) | 9.694 (3.6) | 14.082 (3.6) | 15.162 (3.6) |

Phenylalanine [mg/L] (*p = 0.6375)

| | | | | |
|---|---|---|---|---|
| Baseline: mean (SD) | 8.701 (3.2): | 6.138 (1.3): | 9.376 (1.6): | 7.864 (2.1): |
| Post: mean (SD) | 8.586 (2.1) | 6.460 (1.7) | 9.870 (2.5) | 8.925 (2.1) |

N* = number of participants included in the ITT population,
*p-value for ANOVA (omnibus test)

Blood samples were gathered at the start of the study prior to any treatment and again at the end of the 45 day treatment period. There was no significant effect of treatment within groups, nor was there any significant effect of treatment as compared with baseline for the amino acids tested (Table 20).

TABLE 21
Mineral levels at baseline, and at the end of the study (N = 98)

| | | | |
|---|---|---|---|
| | Bacillus | Bacillus | Probiotic |
| Placebo | clausii | megaterium | cocktail |
| (N* = 24) | (N* = 24) | (N* = 25) | (N* = 25) |

Riboflavin [μg/L] (*p = 0.4222)

| | | | | |
|---|---|---|---|---|
| Baseline: mean (SD) | 452 (16): | 453 (12): | 455 (27): | 453 (13): |
| Post: mean (SD) | 451 (16) | 450 (9) | 450 (12) | 457 (17) |

Nicotinic acid B3 [μg/L] (*p = 0.6467)

| | | | | |
|---|---|---|---|---|
| Baseline: mean (SD) | 7 (0.33): | 7 (0.42): | 8 (0.45): | 7 (0.9): |
| Post: mean (SD) | 7 (0.34) | 7 (0.46) | 7 (0.45) | 7 (0.3) |

Pantoneic acid [μg/L] (*p = 0.8899)

| | | | | |
|---|---|---|---|---|
| Baseline: mean (SD) | 142 (5): | 144 (6): | 146 (4): | 140 (4): |
| Post: mean (SD) | 142 (5) | 143 (6) | 147 (5) | 141 (3) |

Folic acid [μg/L] (*p = 0.5073)

| | | | | |
|---|---|---|---|---|
| Baseline: mean (SD) | 300 (215): | 286 (112): | 377 (164): | 275 (139): |
| Post: mean (SD) | 311 (214) | 283 (112) | 385 (189) | 250 (106) |

N* = number of participants included in the ITT population,
*p-value for ANOVA (omnibus test)

Blood samples were gathered at the start of the study prior to any treatment and again at the end of the 45 day treatment period. There was no significant effect of treatment within groups, nor was there any significant effect of treatment as compared with baseline for mineral levels (Table 21).

### Microbiota Changes

Samples from subjects collected before and after the treatment period were selected for comprehensive microbiota analysis. After removal of short reads and low quality reads, 202,413 sequences were retained, with a mean of 2,736 sequences per sample and an average length of 440 nucleotides. Using the ESPRIT-tree, and after removal of OTUs containing less than 10 sequences, 1,077 and 1,618 OTUs at the 95 and 98% similarity level were retained.

FIG. 28 is a boxplot showing the Chao1 values distribution in each experimental group for Day 1 and Day 45. Dotted lines connect the paired samples. A paired Wilcoxon test was used to compare the distribution of the groups. A p-value less than 0.05 should be considered as statistically significant.

FIG. 29 is a boxplot showing the Chao1 values distribution in each experimental group for Day 1 and Day 45. A Wilcoxon test was used to compare the distribution of each experimental group against the Placebo. A p-value less than 0.05 should be considered as statistically significant.

FIG. 30 illustrates PCoA clustering performed on the Bray-Curtis dissimilarity matrix. Each treatment is separated in a different tab while colours and shape are associated with the time points. Samples from the two time points tend to cluster together for all the treatments, and the data are not significantly different from each other at day 1 baseline readings. Samples were not significantly different from each other as a consequence of treatment within or between groups.

TABLE 22
Proportion of people that had reported symptoms of respiratory
tract infection in Participant diary 1 (N = 123).

| | Bacillus | Bacillus | Bacillus | Probiotic | |
|---|---|---|---|---|---|
| | coagulans | clausii | megaterium | cocktail | Placebo |
| | (N* = 25) | (N* = 24) | (N* = 25) | (N* = 25) | (N* = 24) |
| Fever (%) | 1/25 (4.0) | 2/24 (8.3) | 2/25 (8.0) | 1/25 (4.0) | 2/24 (8.3) |
| Headache (%) | 6/25 (24.0) | 7/24 (29.2) | 8/25 (32.0) | 5/25 (20.0) | 6/24 (25.0) |
| Stuffed nose (%) | 2/25 (8.0) | 2/24 (8.3) | 2/25 (8.0) | 1/25 (4.0) | 3/24 (12.5) |
| Runny nose thick | 0/25 (0.0) | 0/24 (0.0) | 0/25 (0.0) | 3/25 (12.0) | 0/24 (0.0) |
| Runny nose watery (%) | 2/25 (8.0) | 3/24 (12.5) | 5/25 (20.0) | 1/25 (4.0) | 3/24 (12.5) |
| Sore throat (%) | 0/25 (0.0) | 1/24 (4.2) | 5/25 (20.0) | 3/25 (12.0) | 4/24 (16.7) |
| Dry cough (%) | 2/25 (8.0) | 2/24 (8.3) | 2/25 (8.0) | 1/25 (4.0) | 3/24 (12.5) |
| Productive cough (%) | 0/25 (0.0) | 0/24 (0.0) | 1/25 (4.0) | 2/25 (8.0) | 2/24 (8.3) |
| Sore ear (%) | 0/25 (0.0) | 0/24 (0.0) | 0/25 (0.0) | 1/25 (4.0) | 0/24 (0.0) |

N* = number of participants included in the ITT population

A significant difference among treatment groups was detected only in the number of days with runny nose-thick (p*=0.018) probably due the fact that only three participants in Probiotic cocktail group had reported this symptom, while in other four treatment groups none of the participants had reported this symptom. However, further analysis (Mann-Whitney U test with Holm's correction) where number of days with runny nose-thick was compared between Probiotic cocktail group and placebo group, did not show significant differences, probably due to the low sample size.

TABLE 23
Proportion of people with clinically relevant infection
reported in Participant diary 1 (N = 123).

| | Bacillus | Bacillus | Bacillus | Probiotic | |
|---|---|---|---|---|---|
| | coagulans | clausii | megaterium | cocktail | Placebo |
| | (N* = 25) | (N* = 24) | (N* = 25) | (N* = 25) | (N* = 24) |
| Gastrointestinal | 0/25 (0.0) | 0/24 (0.0) | 0/25 (0.0) | 0/25 (0.0) | 2/24 (8.3) |
| infection (%) | | | | | |
| Respiratory tract | 1/25 (4.0) | 2/24 (8.3) | 2/25 (8.0) | 3/25 (12.0) | 6/24 (25.0) |
| infection (%) | | | | | |
| Urinary tract | 1/25 (4.0) | 0/24 (0.0) | 1/25 (4.0) | 2/25 (8.0) | 1/24 (4.2) |
| infection (%) | | | | | |

N* = number of participants included in the ITT population

Kruskal-Wallis test did not show any significant differences in the number of days with clinically relevant infection treatment groups. However, a borderline statistically significant result was observed for clinically relevant gastrointestinal infection. This is probably due the fact that only no participants in the four probiotic treatment groups experienced clinically relevant gastrointestinal infection, while in probiotic group in total 2 days of such infection were observed, which could have happened by chance.

Nevertheless, compared to placebo, none of the study products containing probiotics showed a statistically significant difference.

TABLE 24
Proportion of loose/hard stool per all stools in
weeks 6 and 7 of the treatment period (N = 121)

| | | | | |
|---|---|---|---|---|
| Bacillus | Bacillus | Bacillus | Probiotic | |
| coagulans | clausii | megaterium | cocktail | Placebo |
| (N = 24) | (N = 23) | (N = 25) | (N = 25) | (N = 24) |

Loose stool (p= 0.033)*

| | | | | | |
|---|---|---|---|---|---|
| mean (SD): | 7% (10%): | 4% (14%): | 4% (10%): | 2% (4%): | 8% (19%): |
| min-max | 0%-26% | 0%-50% | 0%-40% | 0%-13% | 0%-91% |
| p-value vs | 0.591 | 0.150 | 0.271 | 0.186 | / |
| placebo | | | | | |

Hard stool (p = 0.528)*

| | | | | | |
|---|---|---|---|---|---|
| mean (SD): | 22% (29%): | 13% (27%): | 9% (16%): | 15% (27%): | 14% (27%): |
| min-max | 0%-100% | 0%-100% | 0%-56% | 0%-100% | 0%-100% |

*p-value for Kruskal-Wallis test. If p < 0.05, individual comparisons with placebo were calculated (Mann-Whitney U test with Holm's correction).

A significant difference among groups was detected in the proportion of loose stools in the total treatment period as well as in weeks 6 and 7 of the treatment period. However, further analysis (Mann-Whitney U test with Holm's correction) did not show significant differences, probably due to the low sample size. The participants in the Probiotic cocktail group had the smallest proportion of loose stool per all stools.

TABLE 25
Proportion of people that had reported symptoms of gastrointestinal
infection in Participant diary 2 (N = 118)

| | Bacillus | Bacillus | Bacillus | Probiotic | |
|---|---|---|---|---|---|
| | coagulans | clausii | megaterium | cocktail | Placebo |
| | (N* = 25) | (N* = 24) | (N* = 25) | (N* = 25) | (N* = 24) |
| Loss of apetite (%) | 0/24 (0.0) | 0/23 (0.0) | 0/23 (0.0) | 0/25 (0.0) | 1/23 (4.3) |
| Diarrhea (%) | 0/24 (0.0) | 1/23 (4.3) | 1/23 (4.3) | 0/25 (0.0) | 1/23 (4.3) |
| Constipation (%) | 0/24 (0.0) | 0/23 (0.0) | 0/23 (0.0) | 0/25 (0.0) | 3/23 (13.0) |
| Vomiting (%) | 0/24 (0.0) | 0/23 (0.0) | 0/23 (0.0) | 0/25 (0.0) | 1/23 (4.3) |
| Gases (%) | 0/24 (0.0) | 1/23 (4.3) | 1/23 (4.3) | 2/25 (8.0) | 2/23 (8.7) |
| Bowel sounds (%) | 0/24 (0.0) | 0/23 (0.0) | 0/23 (0.0) | 0/25 (0.0) | 0/23 (0.0) |
| Cramping/stomach pain (%) | 1/24 (4.2) | 1/23 (4.3) | 0/23 (0.0) | 1/25 (4.0) | 2/23 (8.7) |
| Bloating (%) | 1/24 (4.2) | 1/23 (4.3) | 0/23 (0.0) | 0/25 (0.0) | 1/23 (4.3) |

N* = number of participants included in the ITT population

A significant difference among groups was detected only in the number of days with constipation (p*=0.013) probably due the fact that only three participants in Placebo group had reported this symptom in Participant diary 2, while in other four treatment groups none of the participants had reported this symptom. However, further analysis (Mann-Whitney U test with Holm's correction) where number of days with constipation was compared between individual probiotic group and placebo group did not show significant differences, probably due to low sample size.

This study has addressed the safety and efficacy of new probiotics, namely Bacillus coagulans, Bacillus clausii, Bacillus megaterium and probiotic cocktail containing Bacillus subtilis, Bacillus megaterium, Bacillus clausii and Bacillus coagulans.

The gastrointestinal health of the participants at baseline among the treatment groups did not differ between the study groups, which was expected due to randomization.

The primary outcome of the study (safety) was achieved, as 17 AEs were reported in total with no SAEs. Causality assessment revealed no relation between the reported AEs and the study products.

None of the efficacy related outcomes showed any statistically significant difference, however this comes as no surprise due to small sample size per study group. Still, some trends favouring active products were observed, specifically in the Gut-brain axis scores and proportion of loose stools.

To conclude, probiotic products showed to be safe to use in adults, and have shown some favourable data regarding Gut-brain axis and stool consistency.

### DISCUSSION

The use of Bacillus probiotics in maintenance of gut health has been largely supported in the last years and has driven its clinical applications. Their favorable effects have been linked to several properties, such as antimicrobial and immunomodulatory activity, regulation of cell growth and differentiation, cell-cell signaling, cell adhesion, signal transcription and transduction, production of vitamins and gut protection from genotoxic agents.

This trial was conducted to evaluate the effect of three probiotic treatments on general wellness and gastrointestinal symptoms in healthy adults. There were no safety or tolerability concerns and no adverse events. With this small study cohort in healthy individuals without any gastrointestinal issues, there were no negative effects on stool regularity and consistency, and no negative effects on sadness, irritability, energy, appetite, tension, stress, sleep, cardiovascular events, aches and pains, and dizziness. In fact, we report a decrease in the incidence of loose stools throughout the intervention period attributable to the administration of the probiotic cocktail.

### REFERENCES

Abriouel, H., Franz, C., Omar, N., & Galvez, A. (2011). Diversity and applications of Bacillus bacteriocins. FEMS Microbiology Reviews, 35 (1), 201-232. doi: doi.org/10.1111/j.1574-6976.2010.00244.x
Afrilasari W, Widanarni, Meryadini A (2016) “Effect of Probiotic Bacillus Megaterium PTB 1.4 on the Population of Intestinal Microflora, Digestive Enzyme Activity and the Growth of Catfish (Clarias Sp.).” HAYATI Journal of Biosciences, vol. 23, no. 4, pp. 168-172., doi: 10.1016/j.hjb.2016.12.005.
Alippi, A. (1995). Detection of Bacillus larvae spores in Argentinian honeys by using a semi-selective medium. Microbiology SEM, 11, 343-350.
Alippi A, Reynaldi F, Lopez A, De Giusti M, Aguilar O (2004) “Molecular Epidemiology of Paenibacillus Larvae Larvae and Incidence of American Foulbrood in Argentinean Honeys from Buenos Aires Province. “Journal of Apicultural Research, vol. 43, no. 3 pp. 135-143., doi: 10.1080/00218839.2004.11101124.
Baron, M. (2009). Original Research: A Patented Strain of Bacillus coagulans Increased Immune Response to Viral Challenge. Postgraduate Medicine, 121(2), 114-118. doi: 10.3810/pgm.2009.03.1971
Benno, Y., Suzuki, K., Suzuki, K., Narisawa, K., Bruce, W. R., & Mitsuoka, T. (1986). Comparison of the Fecal Microflora in Rural Japanese and Urban Canadians. Microbiology and Immunology, 30(6), 521-532. doi: 10.1111/j.1348-0421.1986.tb02978.x
Chan, Y. K., Estaki, M., & Gibson, D. L. (2013). Clinical Consequences of Diet-Induced Dysbiosis. Annals of Nutrition and Metabolism, 63(s2), 28-40. doi: 10.1159/000354902
Cutting, S. M. (2011). Bacillus probiotics. Food Microbiology, 28 (2), 214-220. doi: 10.1016/j.fm.2010.03.007
Dolin, B. (2009). Effects of a proprietary Bacillus coagulans preparation on symptoms of diarrhea-predominant irritable bowel syndrome. Methods and Findings in Experimental and Clinical Pharmacology, 31(10), 655. doi: 10.1358/mf.2009.31.10.1441078
Elshaghabec, F. M. F., Rokana, N., Gulhane, R. D., Sharma, C., & Panwar, H. (2017). Bacillus As Potential Probiotics: Status, Concerns, and Future Perspectives. Frontiers in Microbiology, 8. doi: 10.3389/fmicb.2017.01490
Endres, J., Clewell, A., Jade, K., Farber, T., Hauswirth, J., & Schauss, A. (2009). Safety assessment of a proprietary preparation of a novel Probiotic, Bacillus coagulans, as a food ingredient. Food and Chemical Toxicology, 47(6), 1231-1238. doi: 10.1016/j.fct.2009.02.018
Ferreira, C. M. H., Vilas-Boas, Â., Sousa, C. A., Soares, H. M. V. M., & Soares, E. V.
(2019). Comparison of five bacterial strains producing siderophores with ability to chelate iron under alkaline conditions. AMB Express, 9 (1). doi: 10.1186/s13568-019-0796-3
Gabrielli, M., Lauritano, E. C., Scarpellini, E., Lupascu, A., Ojetti, V., Gasbarrini, G., . . . Gasbarrini, A. (2009). Bacillus clausii as a Treatment of Small Intestinal Bacterial Overgrowth. The American Journal of Gastroenterology, 104 (5), 1327-1328. doi: 10.1038/ajg.2009.91
Gepner, Y., Hoffman, J. R., Shemesh, E., Stout, J. R., Church, D. D., Varanoske, A. N., . . . . Ostfeld, I. (2017). Combined effect of Bacillus coagulans GBI-30, 6086 and HMB supplementation on muscle integrity and cytokine response during intense military training. Journal of Applied Physiology, 123 (1), 11-18. doi: 10.1152/japplphysiol.01116.2016
Gilliam, Martha. (1979) “Microbiology Of Pollen And Bee Bread: The Genus Bacillus.” Apidologie, vol. 10, no. 3, pp. 269-274, doi: 10.1051/apido: 19790304.
Gilliam, Martha, and Diane K. Valentine. (1976) “Bacteria Isolated from the Intestinal Contents of Foraging Worker Honey Bees, Apis Mellifera: The Genus Bacillus.” Journal of Invertebrate Pathology, vol. 28, no. 2, pp. 275-276, doi: 10.1016/0022-2011 (76) 90137-3.
Horosheva, T. V., Vodyanoy, V., & Sorokulova, I. (2014). Efficacy of Bacillus probiotics in prevention of antibiotic-associated diarrhoea: a randomized, double-blind, placebo-controlled clinical trial. JMM Case Reports, 1 (3). doi: 10.1099/jmmcr.0.004036
Hun, L. (2009). Original Research: Bacillus coagulans Significantly Improved Abdominal Pain and Bloating in Patients with IBS. Postgraduate Medicine, 121 (2), 119-124. doi: 10.3810/pgm.2009.03.1984
Hyronimus, B., Marrec, C. L., Sassi, A. H., & Deschamps, A. (2000). Acid and bile tolerance of spore-forming lactic acid bacteria. International Journal of Food Microbiology, 61 (2-3), 193-197. doi: 10.1016/s0168-1605(00)00366-4
Ivanovics, G. & Alfoldi, L. (1957). Bacteriocinogenesis in Bacillus megaterium. J. gen. Microbiol. 16, 522
Jensen, G., Cash, H., Farmer, S., & Keller, D. (2017). Inactivated probiotic Bacillus coagulans GBI-30 induces complex immune activating, anti-inflammatory, and regenerative markers in vitro. Journal of Inflammation Research, Volume 10, 107-117. doi: 10.2147/jir.s141660
Kalman, D. S., Schwartz, H. I., Alvarez, P., Feldman, S., Pezzullo, J. C., & Krieger, D. R. (2009). A prospective, randomized, double-blind, placebo-controlled parallel-group dual site trial to evaluate the effects of a Bacillus coagulans-based product on functional intestinal gas symptoms. BMC Gastroenterology, 9 (1). doi: 10.1186/1471-230x-9-85
Kawarizadeh, A., Nojoomi, F., Tabatabaci, M., Hosseinzadeh, S., & Farzanch, M. (2019). The effect of Bacillus coagulans on cytotoxicity and apoptosis induced by Salmonella typhimurium in HT-29 cell culture. Iranian Journal of Microbiology. doi: 10.18502/ijm.v11i4.1468
Keller, D., Dinter, R. V., Cash, H., Farmer, S., & Venema, K. (2017). Bacillus coagulans GBI-30, 6086 increases plant protein digestion in a dynamic, computer-controlled in vitro model of the small intestine (TIM-1). Beneficial Microbes, 8(3), 491-496. doi: 10.3920/bm2016.0196
Khochamit, N., Siripornadulsil, S., Sukon, P., & Siripornadulsil, W. (2015). Antibacterial activity and genotypic—phenotypic characteristics of bacteriocin-producing Bacillus subtilis KKU213: Potential as a probiotic strain. Microbiological Research, 170, 36-50. doi: 10.1016/j.micres.2014.09.004
Kimmel, M., Keller, D., Farmer, S., & Warrino, D. (2010). A controlled clinical trial to evaluate the effect of GanedenBC30 on immunological markers. Methods and Findings in Experimental and Clinical Pharmacology, 32(2), 129. doi: 10.1358/mf.2010.32.2.1423881
Kotb, E. (2014). Purification and partial characterization of serine fibrinolytic enzyme from Bacillus megaterium KSK-07 isolated from kishk, a traditional Egyptian fermented food. Applied Biochemistry and Microbiology, 51(1), 34-43. doi: 10.1134/s000368381501007x
Lakshmi, S. G., Jayanthi, N., Saravanan, M., & Ratna, M. S. (2017). Safety assessment of Bacillus clausii UBBC07, a spore forming probiotic. Toxicology Reports, 4, 62-71. doi: 10.1016/j.toxrep.2016.12.004
Liu, P., Xie, J., Liu, J., & Ouyang, J. (2019). A novel thermostable β-galactosidase from Bacillus coagulans with excellent hydrolysis ability for lactose in whey. Journal of Dairy Science, 102(11), 9740-9748. doi: 10.3168/jds.2019-16654
Marseglia, G. L., Tosca, M., Cirillo, I., Licari, A., Leone, M., Marseglia, A., . . . . Ciprandi, G. (2007). Efficacy of Bacillus clausii spores in the prevention of recurrent respiratory infections in children: a pilot study. Therapeutics and Clinical Risk Management, 3(1), 13-17. doi: 10.2147/tcrm.2007.3.1.13
Mazzoli, A., Donadio, G., Lanzilli, M., Saggese, A., Guarino, A. M., Rivetti, M., . . . . Isticato, R. (2019). Bacillus megaterium SF185 spores exert protective effects against oxidative stress in vivo and in vitro. Scientific Reports, 9(1). doi: 10.1038/s41598-019-48531-4
Neag, M. A., Catincan, A., Muntean, D. M., Pop, M. R., Bocsan, C. I., Botan, E. C., & Buzoianu, A. D. (2020). Probiotic Bacillus Spores Protect Against Acetaminophen Induced Acute Liver Injury in Rats. Nutrients, 12 (3), 632. doi: 10.3390/nu12030632
Nista, E. C., Candelli, M., Cremonini, F., Cazzato, I. A., Zocco, M. A., Franceschi, F., . . . . Gasbarrini, A. (2004). Bacillus clausii therapy to reduce side-effects of anti-Helicobacter pylori treatment: randomized, double-blind, placebo controlled trial. Alimentary Pharmacology and Therapeutics, 20(10), 1181-1188. doi: 10.1111/j.1365-2036.2004.02274.x
Nithya, V., & Halami, P. M. (2012). Evaluation of the probiotic characteristics of Bacillus species isolated from different food sources. Annals of Microbiology, 63 (1), 129-137. doi: 10.1007/s13213-012-0453-4
Padgham, J., and R. Sikora. (2007) “Biological Control Potential and Modes of Action of Bacillus megaterium against Meloidogyne Graminicola on Rice.” Crop Protection, vol. 26, no. 7, pp. 971-977, doi: 10.1016/j.cropro.2006.09.004.
Pelletier, A. & Sygush, J (1990). Purification and characterization of three chitosanase activities from Bacillus megaterium P1. Appl Environ Microbiol 56, 844-848
Ripert, G., Racedo, S. M., Elie, A. M., Jacquot, C., Bressollier, P., & Urdaci, M. C. (2016). Secreted Compounds of the Probiotic Bacillus clausii Strain O/C Inhibit the Cytotoxic Effects Induced by Clostridium difficile and Bacillus cereus Toxins. Antimicrobial Agents and Chemotherapy, 60(6), 3445-3454. doi: 10.1128/aac.02815-15
Rochín-Medina, J. J., Ramírez-Medina, H. K., Rangel-Peraza, J. G., Pineda-Hidalgo, K. V., & Iribe-Arellano, P. (2017). Use of whey as a culture medium for Bacillus clausii for the production of protein hydrolysates with antimicrobial and antioxidant activity. Food Science and Technology International, 24 (1), 35-42. doi: 10.1177/1082013217724705
Salvetti, E., Orrù, L., Capozzi, V, Martina Am Lamontanara A, Keller D, Cash H, Felis G, Cattivelli L, Torriani S, Spano G. (2016) Integrate genome-based assessment of safety for probiotic strains: Bacillus coagulans GBI-30, 6086 as a case study. Appl Microbiol Biotechnol 100:4595. https://doi.org/10.1007/s00253-016-7416-9
Scholle M D, White C A, Kunnimalaiyaan M, Vary P S (2003) “Sequencing and Characterization of pBM400 from Bacillus megaterium Q M B1551.” Applied and Environmental Microbiology, vol. 69, no. 11, pp. 6888-6898, doi: 10.1128/aem.69.11.6888-6898.2003.
Sharma, S., & Kanwar, S. S. (2017). Adherence potential of indigenous lactic acid bacterial isolates obtained from fermented foods of Western Himalayas to intestinal epithelial Caco-2 and HT-29 cell lines. Journal of Food Science and Technology, 54 (11), 3504-3511. doi: 10.1007/s13197-017-2807-1
Snowdon, Jill A, and Dean O Cliver. (1996) “Microorganisms with Honey.” International Journal of Food Microbiology.
Sudha, M., Radkar, N., & Maurya, A. (2012). Effect of Supplementation of Probiotic Bacillus coagulans Unique IS-2 on Hypercholesterolemia Subjects: A Clinical Study. International Journal of Probiotics and Prebiotics, 6 (2).
Sudha, M. R., Yelikar, K. A., & Deshpande, S. (2012). Clinical Study of Bacillus coagulans Unique IS-2 (ATCC PTA-11748) in the Treatment of Patients with Bacterial Vaginosis. Indian Journal of Microbiology, 52 (3), 396-399. doi: 10.1007/s12088-011-0233-2
Sudha, M. R., Bhonagiri, S., & Kumar, M. A. (2013). Efficacy of Bacillus clausii strain UBBC-07 in the treatment of patients suffering from acute diarrhoea. Beneficial Microbes, 4 (2), 211-216. doi: 10.3920/bm2012.0034
Sudha, M. R., Jayanthi, N., Aasin, M., Dhanashri, R., & Anirudh, T. (2018). Efficacy of Bacillus coagulans Unique IS2 in treatment of irritable bowel syndrome in children: a double blind, randomised placebo controlled study. Beneficial Microbes, 9 (4), 563-572. doi: 10.3920/bm2017.0129
Sudha, M. R., Jayanthi, N., Pandey, D., & Verma, A. (2019). Bacillus clausii UBBC-07 reduces severity of diarrhoea in children under 5 years of age: a double blind placebo controlled study. Beneficial Microbes, 10 (2), 149-154. doi: 10.3920/bm2018.0094
Sumathi C, Nandhini A, Padmanaban J (2017) “Antagonistic Activity of Probiotic Bacillus megaterium against Streptococcus mutans.” International Journal of Pharma and Bio Science, vol. 8, no. 1, doi: 10.22376/ijpbs.2017.8.1.p 270-274.
Thakur, K., Tomar, S. K., & De, S. (2016). Lactic acid bacteria as a cell factory for riboflavin production. Microbial Biotechnology, 9 (4), 441-451. doi: 10.1111/1751-7915.12335
Tysset, C., Durand, C., and Taliergio, Y. P. (1970) Contribution to the study of the microbial contamination and the hygiene of commercial honey. Rec. Med. Vet. 146, 1471-1492.
Vary, P. S., Biedendieck, R., Fuerch, T., Meinhardt, F., Rohde, M., Deckwer, W., & Jahn, D. (2007). Bacillus megaterium—from simple soil bacterium to industrial protein production host. Applied Microbiology and Biotechnology, 76 (5), 957-967. doi: 10.1007/s00253-007-1089-3
von Tersch, M. A. and Carlton, B. C. (1983) Bacteriocin from Bacillus megaterium ATCC 19213: comparative studies with megacin A-216. Journal of Bacteriology 155, 866-871.

The invention is not limited to the embodiment described herein but can be amended or modified without departing from the scope of the present invention.

The use of the terms “a,” “an,” “the,” and similar referents in the context of describing the present invention (especially in the context of the claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. Use of the term “about” is intended to describe values either above or below the stated value in a range of approximately ±10%; in other embodiments, the values may range in value above or below the stated value in a range of approximately ±5%; in other embodiments, the values may range in value above or below the stated value in a range of approximately ±2%; in other embodiments, the values may range in value above or below the stated value in a range of approximately ±1%. The preceding ranges are intended to be made clear by context, and no further limitation is implied. All methods described herein can be performed in any suitable order unless otherwise indicated here in or otherwise clearly contradicted by context. The use of any and all examples, or exemplary language (e.g., “such as”) provided herein, is intended merely to better illuminate the invention and does not pose a limitation on the scope of the invention unless otherwise stated. No language in the specification should be construed as indicating any non-claimed element as essential to the practice of the invention.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

All references cited herein are incorporated by reference in their entireties. The present invention may be embodied in other specific forms without departing from the spirit or essential attributes thereof, and, accordingly, reference should be made to the appended claims, rather than to the foregoing specification, as indicating the scope of the invention.

1. A Bacillus clausii strain comprising a purified microbial population that comprises one or more bacteria with a gyrB that shares at least 97% identity with SEQ ID NO: 1;
and/or that comprises one or more bacteria with a 16S rRNA that shares at least 97% identity with SEQ ID NO: 2.

2. The Bacillus clausii strain of claim 1 that shares at least 97% identity with SEQ ID NO: 3.

3. The Bacillus clausii strain of claim 1 wherein the purified microbial population comprises a bacterium with a 16S nucleic acid sequence comprising SEQ ID NO:2.

4. The Bacillus clausii strain of claim 1 wherein the purified microbial population comprises a bacterium with gyrB nucleic acid sequence comprising SEQ ID NO: 1.

5. The Bacillus clausii strain of claim 1 wherein the purified microbial population comprises a bacterium with a 16S nucleic acid sequence comprising SEQ ID NO: 2 and with a gyrB nucleic acid sequence comprising SEQ ID NO: 1.

6. A microbial composition comprising the Bacillus clausii strain of claim 1 together with a comestibly acceptable carrier and/or diluent.

7. The microbial composition of claim 6, wherein a unit dose of the composition comprises 10⁶-10¹³ CFU of the Bacillus clausii strain.

8. The microbial composition of claim 6, further comprising a mucous adherent excipient.

9. The microbial composition of claim 6, further comprising at least one further probiotic Bacillus spp. strain.

10. The microbial composition of claim 6, wherein the microbial composition is formulated as a tablet, a pill, a capsule, a powder, a solution, a suspension, or an emulsion.

11. The microbial composition of claim 6, wherein the microbial composition is formulated as a food.

12. (canceled)

13. A method of treating vaginal infections, urinary tract infections, gastrointestinal diseases, improving immune health, protection against oxidative stress, cleansing and detoxification, improving metabolic health or cardiovascular health, the method comprising the step of administering to a human subject the Bacillus clausii strain of claim 1.

14. (canceled)

15. A method of treating vaginal infections, urinary tract infections, gastrointestinal diseases, improving immune health, protection against oxidative stress, cleansing and detoxification, improving metabolic health or cardiovascular health, the method comprising the step of administering to a human subject the microbial composition of claim 6.

16. A method of improving microbiome within a subject, comprising the step of administering to the subject a composition comprising a probiotic, wherein the probiotic comprises the Bacillus clausii strain of claim 1.

17. (canceled)

18. (canceled)
